SEQUENCE LISTING (56 sequences)

SEQ ID NO: 1 - protein, 14 aa, Artificial Sequence
  Synthetic peptide
KLAKLAKKLAKLAK

SEQ ID NO: 2 - protein, 19 aa, Artificial Sequence
  Synthetic peptide
  MISC_FEATURE (17)..(19): D-amino acid
CGYEQDPWGVRYWYGCKKK

SEQ ID NO: 3 - protein, 16 aa, Artificial Sequence
  Synthetic peptide
  MISC_FEATURE (14)..(16): D-amino acid
CGDPWGVRYWYGCKKK

SEQ ID NO: 4 - protein, 15 aa, Artificial Sequence
  Synthetic peptide
  MISC_FEATURE (13)..(15): D-amino acid
CGDPWGVRYWGCKKK

SEQ ID NO: 5 - protein, 19 aa, Artificial Sequence
  Synthetic peptide
  MISC_FEATURE (1)..(1): N-terminal acetyl moiety
YEQDPWGVKWWYGGGSKKK

SEQ ID NO: 6 - protein, 19 aa, Artificial Sequence
  Synthetic peptide
  MISC_FEATURE (1)..(1): Optional N-terminal functional group
  MISC_FEATURE (10)..(10): D-amino acid
YEQDPWGVKWWYGGGSKKK

SEQ ID NO: 7 - protein, 19 aa, Artificial Sequence
  Synthetic peptide
  MISC_FEATURE (1)..(1): Optional N-terminal functional group
  MISC_FEATURE (10)..(11): D-amino acid
YEQDPWGVKWWYGGGSKKK

SEQ ID NO: 8 - protein, 19 aa, Artificial Sequence
  Synthetic peptide
  MISC_FEATURE (1)..(1): Optional N-terminal functional group
  MISC_FEATURE (17)..(19): D-amino acid
YEQDPWGVKPWYGGGSKKK

SEQ ID NO: 9 - protein, 19 aa, Artificial Sequence
  Synthetic peptide
  MISC_FEATURE (1)..(1): Optional N-terminal functional group
  MISC_FEATURE (17)..(19): D-amino acid
YEQDPWGVKYWYGGGSKKK

SEQ ID NO: 10 - protein, 19 aa, Artificial Sequence
  Synthetic peptide
  MISC_FEATURE (1)..(1): Optional N-terminal functional group
YEQDPWGVRWWYGGGSKKK

SEQ ID NO: 11 - protein, 19 aa, Artificial Sequence
  Synthetic peptide
  MISC_FEATURE (1)..(1): Optional N-terminal functional group
  MISC_FEATURE (17)..(19): D-amino acid
YEQDPWGVRYWYGGGSKKK

SEQ ID NO: 12 - protein, 19 aa, Artificial Sequence
  Synthetic peptide
  MISC_FEATURE (1)..(1): Optional N-terminal functional group
  MISC_FEATURE (10)..(10): Xaa is P, D, T, R, or H
  MISC_FEATURE (17)..(19): D-amino acid
YEQDPWGVKXWYGGGSKKK

SEQ ID NO: 13 - protein, 19 aa, Artificial Sequence
  Synthetic peptide
  MISC_FEATURE (1)..(1): Optional N-terminal functional group
  MISC_FEATURE (12)..(12): D-amino acid
  MISC_FEATURE (17)..(19): D-amino acid
YEQDPWGVRYWYGGGSKKK

SEQ ID NO: 14 - protein, 19 aa, Artificial Sequence
  Synthetic peptide
  MISC_FEATURE (1)..(1): Optional N-terminal functional group
  MISC_FEATURE (5)..(5): Xaa is hydroxyproline
  MISC_FEATURE (12)..(12): D-amino acid
  MISC_FEATURE (17)..(19): D-amino acid
YEQDXWGVRYWYGGGSKKK

SEQ ID NO: 15 - protein, 20 aa, Artificial Sequence
  Synthetic peptide
  MISC_FEATURE (1)..(1): Optional N-terminal functional group
  MISC_FEATURE (13)..(13): D-amino acid
  MISC_FEATURE (18)..(20): D-amino acid
RYEQDPWGVRYWYGGGSKKK

SEQ ID NO: 16 - protein, 19 aa, Artificial Sequence
  Synthetic peptide
  DISULFID (1)..(16)
  MISC_FEATURE (17)..(19): D-amino acid
CGYEQDPWGVRYWYGCKKK

SEQ ID NO: 17 - protein, 16 aa, Artificial Sequence
  Synthetic peptide
  DISULFID (1)..(13)
  MISC_FEATURE (14)..(16): D-amino acid
CGDPWGVRYWYGCKKK

SEQ ID NO: 18 - protein, 15 aa, Artificial Sequence
  Synthetic peptide
  DISULFID (1)..(12)
  MISC_FEATURE (13)..(15): D-amino acid
CGDPWGVRYWGCKKK

SEQ ID NO: 19 - protein, 19 aa, Artificial Sequence
  Synthetic peptide
  MISC_FEATURE (1)..(16): Amide-cyclized
  MISC_FEATURE (17)..(19): D-amino acid
DGYEQDPWGVRYWYGKKKK

SEQ ID NO: 20 - protein, 19 aa, Artificial Sequence
  Synthetic peptide
  MISC_FEATURE (1)..(1): Xaa is azidolysine
  MISC_FEATURE (1)..(16): Triazole-cyclized
  MISC_FEATURE (16)..(16): Xaa is propargylglycine
  MISC_FEATURE (17)..(19): D-amino acid
XGYEQDPWGVRYWYGXKKK

SEQ ID NO: 21 - protein, 19 aa, Artificial Sequence
  Synthetic peptide
  MISC_FEATURE (1)..(16): DFS-cyclized
  MISC_FEATURE (17)..(19): D-amino acid
CGYEQDPWGVRYWYGCKKK

SEQ ID NO: 22 - protein, 19 aa, Artificial Sequence
  Synthetic peptide
  MISC_FEATURE (1)..(16): DFS-cyclized
  MISC_FEATURE (17)..(19): D-amino acid
CGYEQDPWGVRYWYGCKKK

SEQ ID NO: 23 - protein, 19 aa, Artificial Sequence
  Synthetic peptide
  MISC_FEATURE (1)..(16): DFBP-cyclized
  MISC_FEATURE (17)..(19): D-amino acid
CGYEQDPWGVKWWYGCKKK

SEQ ID NO: 24 - protein, 21 aa, Artificial Sequence
  Synthetic peptide
  DISULFID (1)..(18)
  MISC_FEATURE (19)..(21): D-amino acid
CFFGYEQDPWGVRYWYGCKKK

SEQ ID NO: 25 - protein, 21 aa, Artificial Sequence
  Synthetic peptide
  DISULFID (1)..(18)
  MISC_FEATURE (2)..(2): Pentafluorophenylalanine
  MISC_FEATURE (3)..(3): Pentafluorophenylalanine
  MISC_FEATURE (19)..(21): D-amino acid
CFFGYEQDPWGVRYWYGCKKK

SEQ ID NO: 26 - protein, 12 aa, Artificial Sequence
  Synthetic peptide
WPTDHQMLRIPM

SEQ ID NO: 27 - protein, 12 aa, Artificial Sequence
  Synthetic peptide
WPWDPLRISDWL

SEQ ID NO: 28 - protein, 12 aa, Artificial Sequence
  Synthetic peptide
LPWPSDQIILMW

SEQ ID NO: 29 - protein, 12 aa, Artificial Sequence
  Synthetic peptide
TYPSTQWFFAKF

SEQ ID NO: 30 - protein, 12 aa, Artificial Sequence
  Synthetic peptide
YPSSEQLLAWWG

SEQ ID NO: 31 - protein, 12 aa, Artificial Sequence
  Synthetic peptide
FFPSEQVLIAAL

SEQ ID NO: 32 - protein, 12 aa, Artificial Sequence
  Synthetic peptide
ELPSVEQLWDFF

SEQ ID NO: 33 - protein, 12 aa, Artificial Sequence
  Synthetic peptide
NAPSIYDWLATL

SEQ ID NO: 34 - protein, 12 aa, Artificial Sequence
  Synthetic peptide
KLPSPYDLYLFL

SEQ ID NO: 35 - protein, 12 aa, Artificial Sequence
  Synthetic peptide
GLPSSAELERLW

SEQ ID NO: 36 - protein, 12 aa, Artificial Sequence
  Synthetic peptide
LPSSAELLWALR

SEQ ID NO: 37 - protein, 12 aa, Artificial Sequence
  Synthetic peptide
RLPTSMELLAAF

SEQ ID NO: 38 - protein, 12 aa, Artificial Sequence
  Synthetic peptide
TWVSDLDMWLGA

SEQ ID NO: 39 - protein, 12 aa, Artificial Sequence
  Synthetic peptide
SYWVPDIVWAGL

SEQ ID NO: 40 - protein, 30 aa, Artificial Sequence
  Synthetic peptide
YEQDPWGVKWWYGGGSKLAKLAKKLAKLAK

SEQ ID NO: 41 - protein, 47 aa, Artificial Sequence
  Synthetic peptide
YEQDPWGVKWWYGGGSKKKFEPKSGWMTFLEVTGKIAEMLSLLKQYC

SEQ ID NO: 42 - protein, 38 aa, Artificial Sequence
  Synthetic peptide
YEQDPWGVKWWYGGGSMRPEIWIAQELRRIGDEFNAYC

SEQ ID NO: 43 - protein, 25 aa, Artificial Sequence
  Synthetic peptide
YEQDPWGVKWWYGGGSQEHQGQGCH

SEQ ID NO: 44 - protein, 18 aa, Artificial Sequence
  Synthetic peptide
YEQDPWGVKWWYGGGSKK

SEQ ID NO: 45 - protein, 19 aa, Artificial Sequence
  Synthetic peptide
  MISC_FEATURE (1)..(1): N-terminal acetyl moeity
YEQDPWGVKWWYGGGSKKK

SEQ ID NO: 46 - protein, 19 aa, Artificial Sequence
  Synthetic peptide
  MISC_FEATURE (1)..(1): N-terminal acetyl moiety
  MISC_FEATURE (10)..(10): D-amino acid
YEQDPWGVKWWYGGGSKKK

SEQ ID NO: 47 - protein, 19 aa, Artificial Sequence
  Synthetic peptide
  MISC_FEATURE (1)..(1): N-terminal acetyl moiety
  MISC_FEATURE (10)..(10): D-amino acid
  MISC_FEATURE (11)..(11): D-amino acid
YEQDPWGVKWWYGGGSKKK

SEQ ID NO: 48 - protein, 19 aa, Artificial Sequence
  Synthetic peptide
  MISC_FEATURE (1)..(1): N-terminal acetyl moiety
  MISC_FEATURE (17)..(19): D-amino acid
YEQDPWGVKPWYGGGSKKK

SEQ ID NO: 49 - protein, 19 aa, Artificial Sequence
  Synthetic peptide
  MISC_FEATURE (1)..(1): N-terminal acetyl moiety
  MISC_FEATURE (17)..(19): D-amino acid
YEQDPWGVKYWYGGGSKKK

SEQ ID NO: 50 - protein, 19 aa, Artificial Sequence
  Synthetic peptide
  MISC_FEATURE (1)..(1): N-terminal acetyl moiety
YEQDPWGVRWWYGGGSKKK

SEQ ID NO: 51 - protein, 19 aa, Artificial Sequence
  Synthetic peptide
  MISC_FEATURE (1)..(1): N-terminal acetyl moiety
  MISC_FEATURE (17)..(19): D-amino acid
YEQDPWGVRYWYGGGSKKK

SEQ ID NO: 52 - protein, 19 aa, Artificial Sequence
  Synthetic peptide
  MISC_FEATURE (1)..(1): N-terminal acetyl moiety
  MISC_FEATURE (10)..(10): Xaa is P, D, T, R, or H
  MISC_FEATURE (17)..(19): D-amino acid
YEQDPWGVKXWYGGGSKKK

SEQ ID NO: 53 - protein, 19 aa, Artificial Sequence
  Synthetic peptide
  MISC_FEATURE (1)..(1): N-terminal acetyl moiety
  MISC_FEATURE (12)..(12): D-amino acid
  MISC_FEATURE (17)..(19): D-amino acid
YEQDPWGVRYWYGGGSKKK

SEQ ID NO: 54 - protein, 19 aa, Artificial Sequence
  Synthetic peptide
  MISC_FEATURE (1)..(1): N-terminal acetyl moiety
  MISC_FEATURE (5)..(5): Xaa is hydroxyproline
  MISC_FEATURE (12)..(12): D-amino acid
  MISC_FEATURE (17)..(19): D-amino acid
YEQDXWGVRYWYGGGSKKK

SEQ ID NO: 55 - protein, 20 aa, Artificial Sequence
  Synthetic peptide
  MISC_FEATURE (1)..(1): N-terminal acetyl moiety
  MISC_FEATURE (13)..(13): D-amino acid
  MISC_FEATURE (18)..(20): D-amino acid
RYEQDPWGVRYWYGGGSKKK

SEQ ID NO: 56 - protein, 19 aa, Artificial Sequence
  Synthetic peptide
  MISC_FEATURE (17)..(19): D-amino acid
CGYEQDPWGVRYWYGCKKK

(19) United States
(12) Patent Application Publication
(10) Pub. No.: US 2017/0246311 A1
(43) Pub. Date: Aug. 31, 2017

(54) PEPTIDES FOR BINDING ALTERNATIVELY ACTIVATED MACROPHAGES

(71) Applicant: University of Washington, Seattle, WA (US)

(72) Inventors: Suzie H. PUN, Seattle, WA (US); Mary Elise CIESLEWICZ, Seattle, WA (US); Chayanon NGAMBENJAWONG, Seattle, WA (US)

(21) Appl. No.: 15/444,301

(22) Filed: Feb. 27, 2017

Related U.S. Application Data
(60) Provisional application No. 62/300,529, filed on Feb. 26, 2016.

(51) Int. Cl. A61K 47/48; C07K 7/64; C08F 8/30; C07K 7/08; C07K 14/00; A61K 38/12

(52) U.S. Cl. CPC A61K 47/48246; C07K 7/08; C07K 7/64; C07K 14/00; A61K 47/48176; C08F 8/30; C08F 2810/40

(57) ABSTRACT

M2 macrophage-binding peptides and polymers and related methods of use are described.

### CROSS REFERENCE TO RELATED APPLICATIONS

This application claims priority to U.S. Provisional Patent Application Ser. No. 62/300,529, filed Feb. 26, 2016, the contents of which are hereby incorporated by reference in their entirety.

### STATEMENT REGARDING FEDERALLY FUNDED RESEARCH

This invention was made with U.S. government support under 1R01CA177272, awarded by the National Institutes of Health. The U.S. Government has certain rights in the invention.

### BACKGROUND OF THE INVENTION

Field of the Invention

The present disclosure relates to molecules and methods for binding to M2 macrophages and related methods of treating cancer. More specifically, the invention relates to M2 macrophage-targeting peptides and biocompatible polymers to which a plurality of M2 macrophage-targeting peptides are bound. The polymers can be used to present a multivalent display of, for example, M2 macrophage-targeting peptides that can further deliver a therapeutic payload.

Description of the Related Art

Despite advancement in medical research, cancer remains one of the leading causes of death in the United States [1]. One of the main limitations in cancer therapies is non-specific side effects, which significantly compromise patients' quality of life and restrict the effective dosage that can be administered [2]. Improvement in nanotechnology and molecular biology enables development of therapeutics that can more precisely target the diseased organs or cells, thus alleviating the side effects [3]. Ligand-mediated active targeting is a powerful strategy used to enhance cellular internalization, and hence potency, of drug-loaded nanoparticles or biomacromolecular therapeutics to cells overexpressing the corresponding receptors [2-4]. This active targeting strategy is used in ligand-decorated nanoparticles that are progressing in different stages of clinical trials as well as the recent FDA approvals of antibody-drug conjugates; Brentuximab vedotin and Trastuzumab emtansine [3,5,6].

The increased use of active targeting strategies for drug delivery or imaging applications especially in cancer is in part due to advances in molecular engineering and combinatorial screening techniques that have led to new targeting ligands for different cell targets [7,8]. Antibodies, proteins, oligonucleotides, peptides, and small molecules have all been successfully used as targeting moieties in several targeted drug delivery systems. The inherent advantages and disadvantages of each of these ligand classes has been reviewed in recent literature [3,8-10]. For tumor targeting applications, peptide targeting ligands offer significant advantages over antibodies due to their smaller size which can enhance tumor penetration, in addition to their lower immunogenicity and higher scalability. Small molecule screening is usually more tedious and less successful compared to the discovery of targeting peptides, which is greatly accelerated by robust phage display screening technology. Moreover, diverse functional groups of amino acids also allow for more flexible conjugation chemistry to cargo of interest. Nonetheless, using peptides as targeting moiety may have disadvantages from lower target affinity and serum stability, which in several cases have been successfully improved via multivalent display of the peptides and rational modifications respectively [8,11-13].

### SUMMARY OF THE INVENTION

The invention meets these needs and others by providing M2 macrophage-binding peptides (M2peps) and methods related thereto.

In one aspect, the disclosure provides a peptide comprising contiguous amino acid residues according to the formula:

X0-X1-D-P*-W-X2-X3-X4-X5-W*-X6-X7

wherein

X0 is absent or an N-terminal functional group;

X1 is 1-7 contiguous amino acid residues;

P* is P or hydroxyproline;

X2 is a single amino acid;

X3 is a single amino acid;

X4 is a single amino acid;

X5 is a single amino acid;

W* is W or w;

X6 is 1-6 contiguous amino acid residues; and

X7 is absent, KKK, or kkk,

wherein when X1 is YEQ, X0 is an N-terminal functional group;
wherein the peptide is linear or is a cyclic peptide; and
   wherein when the peptide is a cyclic peptide, one residue of X1 is covalently bound to one residue of X6, optionally through a linker L.

In one embodiment of the above aspect, the peptide may be cyclic. In another embodiment, the peptide may be acetylated at the N-terminus.

In a second aspect, the disclosure provides a peptide comprising contiguous amino acid residues according to the formula:

X91-X92-X93-X94-X95-X96-X97-X98-X99

wherein

X91 is 0-2 contiguous amino acid residues;

X92 is a single amino acid;

X93 is a single amino acid;

X94 is 0-1 contiguous amino acid residues;

X95 is a single amino acid;

X96 is a single amino acid;

X97 is 0-4 contiguous amino acid residues;

X98 is a single amino acid; and

X99 is 0-5 contiguous amino acid residues; and

and wherein at least one of the following applies:

X92 is W, X93 is P, and X96 is D;

X93 is P, X95 is S, and X98 is A;

X93 is P, X95 is S, and X98 is L; or

X92 is W, X93 is V, X96 is D, and X98 is W.

In one embodiment of the second aspect of the disclosure, the peptide comprises contiguous amino acid residues according to the formula:

X11-W-P-X12-D-X13-X14-X15

wherein

X11 is 0-2 contiguous amino acid residues;

X12 is a single amino acid;

X13 is 0-3 contiguous amino acid residues;

X14 is a single amino acid; and

X15 is 0-4 contiguous amino acid residues.

In another embodiment of the second aspect of the disclosure, the peptide comprises contiguous amino acid residues according to the formula:

X21-X22-P-X23-S-X24-X25-X26-X27-X28

wherein

X21 is 0-1 contiguous amino acid residues;

X22 is a single amino acid;

X23 is 0-1 contiguous amino acid residues;

X24 is a single amino acid;

X25 is a single amino acid;

X26 is 0-3 contiguous amino acid residues;

X27 is a single amino acid; and

X28 is 0-5 contiguous amino acid residues.

In a further embodiment of the second aspect of the disclosure, the peptide comprises contiguous amino acid residues according to the formula:

X41-W-V-X42-D-X43-W-X44

wherein

X41 is 0-2 contiguous amino acid residues;

X42 is a single amino acid;

X43 is 0-3 contiguous amino acid residues; and

X44 is 0-3 contiguous amino acid residues.

In a third aspect of the disclosure, the peptides may be coupled to a therapeutic agent.

In a fourth aspect, the disclosure provides an M2 macrophage-binding agent comprising a peptide of any of the prior aspects, including wherein the peptide is within a loop region of a protein, in a chimeric antigen receptor, or in a viral loop.

In a fifth aspect, the disclosure provides nucleic acids encoding the peptides and binding agents of the prior aspects, recombinant expression vectors comprising the nucleic acids linked to a suitable control sequence, and host cells comprising the recombinant expression vectors.

In a sixth aspect, the disclosure provides pharmaceutical compositions comprising the peptides and binding agents of the prior aspects and a pharmaceutically acceptable carrier.

In a seventh aspect, the disclosure provides a polymer comprising two or more repeating units that form a backbone, wherein the repeating units comprise at least one unit comprising a conjugated peptide of the prior aspects of the disclosure, and optionally one or more unconjugated units.

In one embodiment of the seventh aspect of the disclosure, the polymer further comprises a therapeutic molecule coupled to the polymer. In another embodiment of the seventh aspect of the invention, the polymer further comprises an imaging agent bound to the polymer.

In an eighth aspect, the disclosure provides a method of treating or ameliorating a subject suffering from cancer comprising administering a therapeutically effective dose of a peptide or a polymer of the prior aspects of the disclosure, wherein the peptide or polymer are coupled to a therapeutic agent, thereby delivering the therapeutic agent and treating or ameliorating the cancer.

### BRIEF DESCRIPTION OF THE FIGURES

FIG. 1. (A) MALDI-TOF MS spectra of M2pepBiotin at different serum incubation times. (B) Zoomed-in MALDI-TOF MS spectra of M2pepBiotin at 1 and 2 h. (C) MALDI-TOF MS spectra of AcM2pepBiotin at different serum incubation times. Adjacent to the primary peaks are their respective Na+ adducts. (D) Binding of AcM2pepBiotin versus M2pepBiotin to M1 and M2 macrophages. Stars denote statistical significance between M1 and M2 macrophages in the same treatment group. * P<0.05, ns=not statistically significant.

FIG. 2. (A) MALDI-TOF MS spectra of W10w analogs at different serum incubation times. Adjacent to the primary peaks are their respective Na+ adducts. (B) Binding of M2pepBiotin compared to W10w and W(10,11)w analogs to M1 and M2 macrophages. Unless labeled in pairs, stars denote statistical significance between M1 and M2 macrophages in the same treatment group. * P<0.05.

FIG. 3. MALDI-TOF MS spectra of M2pep analogs: (A) W10P, (B) W10Y, and (C) K9R at different serum incubation times. Adjacent to the primary peaks are their respective Na+ adducts. (D) Binding of the M2pep analogs to M1 and M2 macrophages. Unless labeled in pairs, stars denote statistical significance between M1 and M2 macrophages in the same treatment group. * P<0.05.

FIG. 4. (A) MALDI-TOF MS spectra of cyclic M2pep(RY)Biotin at different serum incubation times. (B) Zoomed-in MALDI-TOF MS spectra of cyclic M2pep(RY)Biotin at 48 h. (C) Binding of cyclic M2pep(RY)Biotin and AcM2pep(RY)Biotin to M1 and M2 macrophages. (D) Binding of cyclic M2pep(RY)Biotin, cRYWY, and cRYW to M1 and M2 macrophages. Unless labeled in pairs, stars denote statistical significance between M1 and M2 macrophages in the same treatment group. * P<0.05, ns=not statistically significant.

FIG. 5. (A) MALDI-TOF MS spectra of Y12y analog at different serum incubation times. (B) Binding of AcM2pep(RY) and Y12y to M1 and M2 macrophages. (C) Binding study Y12y, P5Hyp, and R0 analogs to M1 and M2 macrophages. Stars denote statistical significance between M1 and M2 macrophages in the same treatment group. * P<0.05, ns=not statistically significant.

FIG. 6. M2 macrophage-binding study of M2pepBiotin, AcM2pep(RY)Biotin, and cyclic M2pep(RY)Biotin pre-incubated in serum for different durations. * P<0.05.

FIG. 7. In vivo biodistribution study of M2pep-sulfoCy5 versus cyclic M2pep(RY)-sulfoCy5 in (A) CT-26 tumor model (n=5) and (B) 4T1 tumor model (n=3); (i) Representative xenogen images of the harvested organs (Δ indicates the corresponding pair of the tumor images and the scale bar), (ii) Quantified fluorescence intensity in each organ (Stars denote statistical significance relative to PBS and M2pep-sulfoCy5-treated groups. P<0.05), and (iii) Intratumoral accumulation in CD11b− cells, M1-like TAMs, and M2-like TAMs. Unless labeled in pairs, stars denote statistical significance relative to both CD11b− cells and M1-like TAMs in the same treatment group. * P<0.05.

FIG. 8. (A) Binding of AcM2pep(RY)Biotin, K9R and W10Y analogs to M1 and M2 macrophages. (B) MALDI-TOF MS spectra of AcM2pep(RY)Biotin at different serum incubation times. Adjacent to the primary peaks are their respective Na+ adducts. Unless labeled in pairs, stars denote statistical significance between M1 and M2 macrophages in the same treatment group. * P<0.05, ns=not statistically significant.

FIG. 9. MALDI-TOF MS spectra of crude W10(P,D,T,R,H) analog at different serum incubation times. The crude mixture was synthesized using the split-and-mix strategy at the W10 position. Briefly, YGGGSkkk(K-Biotin) was synthesized on the automatic peptide synthesizer. The peptide on resin was then split into 5 portions, separately coupled with Pro, Asp, Thr, Arg, and His, manually pooled together, and put on the peptide synthesizer to continue the synthesis of the remaining sequence. The crude mixture was used without RP-HPLC purification.

FIG. 10. Schematics elucidating the gating strategy for analysis of intratumoral biodistribution of M2pep analogs in 1) CD11b− population, 2) M1-likeTAMs, and 3) M2-like TAMs.

FIG. 11. Biotin-labeled polymer displaying M2 macrophage-binding peptides was prepared by first synthesizing a copolymer of hydroxypropylmethacrylamide with aminopropylmethacrylamide (poly(HPMA-co-APMA)) and then grafting M2 macrophage binding peptide (called M2pep). A binding study with the polymer was done by detection of biotin on the polymer with streptavidin FITC. The biotin-labeled peptide-grafted polymer was able to mediate selective binding to M2 macrophages over M1 macrophages even at the sub-micromolar concentrations. The polymeric display of M2pep resulted in higher binding even compared to a tetravalent M2pep synthesized by solid phase peptide chemistry.

FIG. 12. Binding curves of M2pep analogs on (A) M2 macrophages and (B) M1 macrophages.

FIG. 13. Binding curves of DFBP-cyclized M2pep(RY)Biotin and DFBP-cyclized M2pepBiotin with M1 and M2 macrophages.

FIG. 14. (A) DFBP-functionalized linear control peptide. (B) Binding study of DFBP-functionalized linear control peptide on M1 and M2 macrophages. Stars denote statistical significance between M2 and M1 macrophages. *P<0.05. (C) Binding study of DFBP-cyclized M2pep(RY)Biotin in PBS and PBSA on M2 macrophages.

FIG. 15. (A) FF- and F(F5)F(F5)-cyclized M2pep(RY)Biotin. (B) Binding curves of DFBP-, FF-, and F(F5)F(F5)-cyclized M2pep(RY)Biotin with M1 and M2 macrophages. (C) Enlarged binding curves at the lower concentration range.

FIG. 16. CD spectra of (A) M2pepBiotin and Disulfide-cyclized M2pep(RY)Biotin, (B) Amide- and Triazolecyclized M2pep(RY)Biotin, (C) DFBP- and DFS-cyclized M2pep(RY)Biotin, and (D) DFBP-cyclized M2pepBiotin and FF-cyclized M2pep(RY)Biotin.

FIG. 17. Serum stability of (A) Amide-, (B) Triazole-, (C) DFBP-, and (D) DFS-cyclized M2pep(RY)Biotin. Left: MALDI-ToF MS spectra. Middle: Selected enlarged regions of the spectra. Right: Identified degraded peptide fragments.

### DETAILED DESCRIPTION OF THE INVENTION

As used herein, the singular forms “a”, “an” and “the” include plural referents unless the context clearly dictates otherwise. “And” as used herein is interchangeably used with “or” unless expressly stated otherwise.

All common terms between different aspects and embodiments of the invention have the same meaning unless the context clearly dictates otherwise. All scientific and technical terms used in this application have meanings commonly used in the art unless otherwise specified.

Unless clearly indicated otherwise by the context, embodiments disclosed for one aspect of the invention can be used in other aspects of the invention as well, and/or in combination with embodiments disclosed in the same or other aspects of the invention. Thus, the disclosure is intended to include, and the invention includes, such combinations, even where such combinations have not been explicitly delineated.

As used throughout the present application, the term “peptide” is used in its broadest sense to refer to a sequence of subunit amino acids, whether naturally occurring or of synthetic origin, and include analogs of naturally occurring amino acids. The peptides of the invention may comprise L-amino acids, D-amino acids (which are resistant to L-amino acid-specific proteases in vivo), or a combination of D- and L-amino acids. The amino acids of the peptides of the invention may substituted for known analogs including, as a non-limiting example, the substitution of one or more tryptophans with analogs such as methyltryptophan, bromo tryptophan, fluorotryptophan. The peptides described herein may be chemically synthesized or recombinantly expressed. The peptides may be linked to other compounds to promote an increased half-life in vivo, such as by PEGylation, HESylation, PASylation, or glycosylation. Such linkage can be covalent or non-covalent as is understood by those of skill in the art. The peptides may be linked to any other suitable linkers, including but not limited to any linkers that can be used for purification or detection (such as FLAG or His tags).

As used herein, amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V). Small (i.e., lowercase) letters denote D-amino acids.

As used herein, “M2 macrophage-binding” and “M2pep,” which can be used interchangeably, mean a peptide that binds M2 macrophages with a higher affinity than it binds to other targets. In certain embodiments, the M2 macrophage-binding peptides bind to M2 macrophages with higher affinity than to other types of macrophages.

The term “nucleic acid” or “polynucleotide” refers to a deoxyribonucleotide or ribonucleotide polymer in either single- or double-stranded form, and unless otherwise limited, encompasses known analogs of natural nucleotides that hybridize to nucleic acids in a manner similar to naturally-occurring nucleotides.

An “isolated” peptide is one that is removed from its original environment. For example, a naturally occurring protein is isolated if it is separated from some or all of the coexisting materials in the natural system. In certain embodiments, the isolated peptide represents 5%, 10%, 20%, 40%, 50%, 60%, 70%, 80%, 90%, 95%, 96%, 97%, 98%, 99% or more of the total peptide content in a sample comprising the isolated peptide. Preferably, such peptides are at least about 90% pure, more preferably at least about 95% pure and most preferably at least about 99% pure. Such isolated peptides can be coupled to other peptides through, for example, chemical conjugations or as part of a fusion peptide, without affecting the purity or portion of the isolated peptide in a sample.

The peptides disclosed herein may be cyclic, they may be acetylated at the N-terminus, and/or they may be coupled to therapeutic molecule. Such therapeutic molecules include, but are not limited to therapeutic peptides. Such therapeutic peptides include, but are not limited to, inflammatory peptides and a pro-apoptotic peptides such as KLA (KLAKLAKKLAKLAK; SEQ ID NO:01).

In one aspect, invention provides an M2 macrophage-binding peptide comprising contiguous amino acid residues according to the formula:

X0-X1-D-P*-W-X2-X3-X4-X5-W*-X6-X7

wherein

X0 is absent or an N-terminal functional group;

X1 is 1-7 contiguous amino acid residues;

P* is P or hydroxyproline;

X2 is a single amino acid;

X3 is a single amino acid;

X4 is a single amino acid;

X5 is a single amino acid;

W* is W or w;

X6 is 1-6 contiguous amino acid residues; and

X7 is absent, KKK, or kkk,

wherein when X1 is YEQ, X0 is an N-terminal functional group;
wherein the peptide is linear or is a cyclic peptide; and
wherein when the peptide is a cyclic peptide, one residue of X1 is covalently bound to one residue of X6, optionally through a linker L.

In one embodiment of this aspect of the invention, X1 comprises YEQ. In another embodiment, P* is P. In a further embodiment, X2 and X3 together are GV. In an additional embodiment, X4 is K or R. In another embodiment, X5 is W, w, Y, or a tyrosine analog, including a halogen-substituted tyrosine analog. Such halogen-substituted tyrosine analogs include, but are not limited to, diiodotyrosine. In a further embodiment, X6 is YGGGS, yGGGS, or YGC.

In another embodiment of this aspect of the invention, the peptide may be cyclic, wherein two residues are bound together through a linker. Exemplary linkers include perfluoroaryl moieties, such as a decafluorobiphenyl moiety or a decafluorobiphenylsulfone moiety, a triazole linker, and an amide linker. In certain embodiments, the peptide is cyclic, and the N-terminal residue of X1 is covalently bound to the C-terminal residue of X6. In some embodiments, X1 comprises lysine and X6 comprises aspartic acid and/or glutamic acid, or vice versa. In some embodiments, X1 comprises an amino acid having an azide moiety and X6 comprises an amino acid having an alkyne moiety, or vice versa. In other embodiments, X1 and X6 each comprise a cysteine residue, and in still further embodiments, a cysteine residue of X1 is covalently bound to a cysteine residue of X6 through a linker. In certain embodiments, the cyclic M2pep may be CGYEQDPWGVRYWYGCkkk (SEQ ID NO:02), CGDPWGVRYWYGCkkk (SEQ ID NO:03), or CGDPWGVRYWGCkkk(SEQ ID NO:04).

In a second aspect, invention provides an M2 macrophage-binding peptide comprising contiguous amino acid residues according to the formula:

X91-X92-X93-X94-X95-X96-X97-X98-X99

wherein

X91 is 0-2 contiguous amino acid residues;

X92 is a single amino acid;

X93 is a single amino acid;

X94 is 0-1 contiguous amino acid residues;

X95 is a single amino acid;

X96 is a single amino acid;

X97 is 0-4 contiguous amino acid residues;

X98 is a single amino acid; and

X99 is 0-5 contiguous amino acid residues; and

and wherein at least one of the following applies:

X92 is W, X93 is P, and X96 is D;

X93 is P, X95 is S, and X98 is A;

X93 is P, X95 is S, and X98 is L; or

X92 is W, X93 is V, X96 is D, and X98 is W.

In certain embodiments of this aspect of the invention, X92 is W. In certain embodiments, X93 is P or V. In further embodiments, X 94 is absent. In another embodiment, X95 is S. In a further embodiment, X96 is D, and in an additional embodiment, X98 is A, L, or W.

In another embodiment of this aspect of the invention is provided an M2 macrophage-binding peptide comprising contiguous amino acid residues according to the formula:

X11-W-P-X12-D-X13-X14-X15

wherein

X11 is 0-2 contiguous amino acid residues;

X12 is a single amino acid;

X13 is 0-3 contiguous amino acid residues;

X14 is a single amino acid; and

X15 is 0-4 contiguous amino acid residues.

In certain embodiments, X11 is absent or is LP. In other embodiments, X12 is T, W, or S; X13 is HQM, PLR, or QII; X14 is L or I; and/or X15 is RIPM, SDWL, or MW.

In a further embodiment of this aspect of the invention is provided an M2 macrophage-binding peptide comprising contiguous amino acid residues according to the formula

X21-X22-P-X23-S-X24-X25-X26-X27-X28

wherein

X21 is 0-1 contiguous amino acid residues;

X22 is a single amino acid;

X23 is 0-1 contiguous amino acid residues;

X24 is a single amino acid;

X25 is a single amino acid;

X26 is 0-3 contiguous amino acid residues;

X27 is a single amino acid; and

X28 is 0-5 contiguous amino acid residues.

In certain embodiments, X21 is T, F, E, N, K, G, or R. In other embodiments, X22 is Y, F, L, or A; X23 is absent or T; X24 is T, S, E, V, I, P, or M; X25 is Q, E, Y, or A; X26 is WFF, QLL, VLI, Q, DW, D, E, or L; X27 is A or L; and/or X28 is KF, WWG, AL, WDFF, ATL, YLFL, ERLW, LWALR, or AAF.

In a further embodiment of this aspect of the invention is provided an M2 macrophage-binding peptide comprising contiguous amino acid residues according to the formula

X41-W-V-X42-D-X43-W-X44

wherein

X41 is 0-2 contiguous amino acid residues;

X42 is a single amino acid;

X43 is 0-3 contiguous amino acid residues; and

X44 is 0-3 contiguous amino acid residues.

In certain embodiments, X41 is T or SY. In further embodiments, X42 is S or P; X43 is LDM or IV; and/or X44 is LGA or AGL.

In further embodiments of the first aspect of the invention, the peptide comprises an amino acid sequence selected from the group consisting of:

(SEQ ID NO: 05)
Ac-YEQDPWGVKWWYGGGSKKK;
(SEQ ID NO: 06)
X0-YEQDPWGVKwWYGGGSKKK;
(SEQ ID NO: 07)
X0-YEQDPWGVKwwYGGGSKKK;
(SEQ ID NO: 08)
X0-YEQDPWGVKPWYGGGSkkk;
(SEQ ID NO: 09)
X0-YEQDPWGVKYWYGGGSkkk;
(SEQ ID NO: 10)
X0-YEQDPWGVRWWYGGGSKKK;
(SEQ ID NO: 11)
X0-YEQDPWGVRYWYGGGSkkk;
(SEQ ID NO: 12)
X0-YEQDPWGVKZaWYGGGSkkk;
(SEQ ID NO: 13)
X0-YEQDPWGVRYWyGGGSkkk;
(SEQ ID NO: 14)
X0-YEQDZbWGVRYWyGGGSkkk;
and
(SEQ ID NO: 15)
X0-RYEQDPWGVRYWyGGGSkkk;

wherein Ac is an acetyl moiety, Za is P, D, T, R, or H, Zb is hydroxyproline, and X0 is as defined above.

In additional embodiments of the first aspect of the invention, the peptide comprises an amino acid sequence selected from the group consisting of:

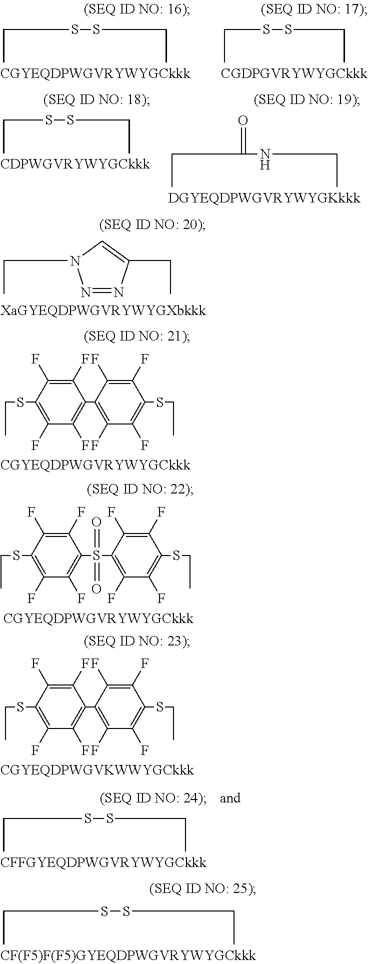

wherein Xa is azidolysine, Xb is propargylglycine; and F(F5) is pentafluorophenylalanine.

In additional embodiments of the second aspect of the invention, the peptide comprises an amino acid sequence selected from the group consisting of:

(SEQ ID NO: 26)
WPTDHQMLRIPM;
(SEQ ID NO: 27)
WPWDPLRISDWL;
(SEQ ID NO: 28)
LPWPSDQIILMW;
(SEQ ID NO: 29)
TYPSTQWFFAKF;
(SEQ ID NO: 30)
YPSSEQLLAWWG;
(SEQ ID NO: 31)
FFPSEQVLIAAL;
(SEQ ID NO: 32)
ELPSVEQLWDFF;
(SEQ ID NO: 33)
NAPSIYDWLATL;
(SEQ ID NO: 34)
KLPSPYDLYLFL;
(SEQ ID NO: 35)
GLPSSAELERLW;
(SEQ ID NO: 36)
LPSSAELLWALR;
(SEQ ID NO: 37)
RLPTSMELLAAF;
(SEQ ID NO: 38)
TWVSDLDMWLGA;
and
(SEQ ID NO: 39)
SYWVPDIVWAGL.

In a further embodiment, the invention provides an isolated peptide comprising or consisting of contiguous amino acids according to the general formula Xm-D-P-W-Xn-Xo-Xp-Xq-W-Xr-Xs, wherein

Xm is 0-4 contiguous amino acid residues;

Xn is any single amino acid;

Xo is any single amino acid;

Xp is any single amino acid;

Xq is any single amino acid;

Xr is any 0-6 contiguous amino acid residues; and

Xs is absent, comprises KKK, or comprises (k)(k)(k), and

wherein at least one of the following applies:
Xq is selected from the amino acids consisting of w, Y, D, R, and H; and
Xp is R.

In the above embodiment, the peptide may be linear or cyclic and when cyclic, may be coupled in the same manner as any of the other cyclic peptides of the invention. In certain instances of the above embodiment, Xp is K. In certain instances of the above embodiment, Xm comprises a cysteine and Xr comprises a cysteine. In certain instances of the above embodiment, Xm comprises or consists of the amino acid sequence YEQ. In other instances of the above embodiment, Xr comprises a C-terminal (y). In still further instances of the above embodiment, Xm comprises lysine and Xr comprises aspartic acid and/or glutamic acid; Xm comprises aspartic acid and/or glutamic acid and Xr comprises lysine; Xm comprises an amino acid having an azide moiety and Xr comprises an amino acid having an alkyne moiety; Xm comprises an amino acid having an alkyne moiety and Xr comprises an amino acid having an azide moiety; Xm comprises cysteine-diphenylalanine and Xr comprises cysteine.

In a certain aspect, the invention further comprises a therapeutic molecule, such as a proapoptotic peptide coupled to the peptide. Exemplary coupled peptides of the invention include:

| Name | Structure |
|---|---|
| M2pep-KLA | YEQDPWGVKWWY-GGGS-KLAKLAKKLAKLAK |
| | (SEQ ID NO: 40) |
| [M2pep]₂-KLA | 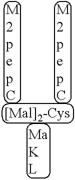 |
| [M2pep]₂- [KLA]₂ | 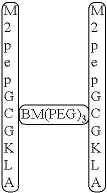 |
| M2pep-KLA- polymer | 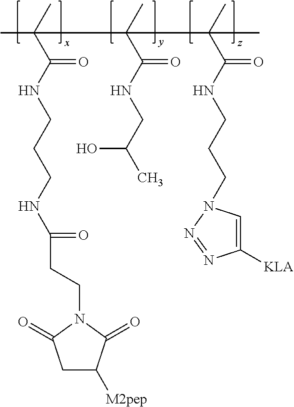 |
| | X = 5-10 |
| | Y = 100-400 |
| | Z = 5-10 |
| | KLA peptide may be substituted with any pro-apoptotic peptides |
| M2pep-ATAP | YEQDPWGVKWWY-GGGS-KK- |
| | KFEPKSGWMTFLEVTGKIAEMLSLLKQYC |
| | (SEQ ID NO: 41) |
| M2pep-BIM | YEQDPWGVKWWY-GGGS- |
| | MRPEIWIAQELRRIGDEFNAYC |
| | (SEQ ID NO: 42) |
| M2pep- | YEQDPWGVKWWY-GGGS-QEHQGQGCH |
| ARTS(266- | (SEQ ID NO: 43) |
| 274) | |
| M2pep- inhibitor- polymer | 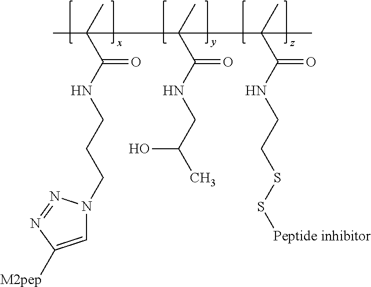 |
| | X = 5-10 |
| | Y = 100-400 |
| | Z = 5-20 |
| M2pep-HIF-1 inhibitor- polymer | 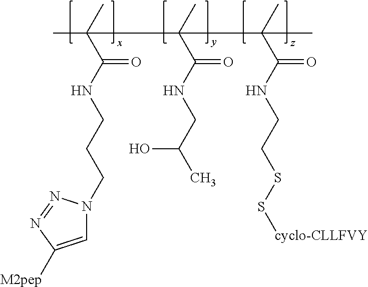 |
| | X = 5-10 |
| | Y = 100-400 |
| | Z = 5-20 |
| M2pep- doxorubicin- polymer | 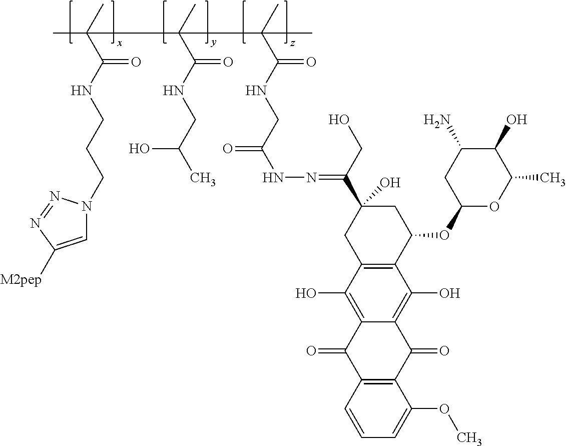 |
| | X = 5-10 |
| | Y = 100-400 |
| | Z = 50-100 |
| | Doxorubicin may be substituted with alternatives of similar pharmacological |
| | activities. |
| M2pep- dexamethasone- polymer | 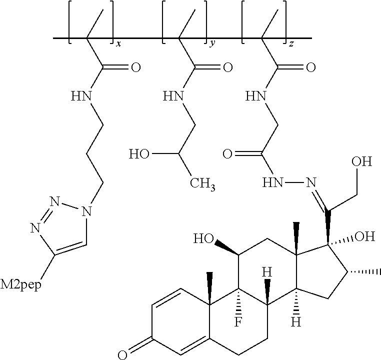 |
| | X = 5-10 |
| | Y = 100-400 |
| | Z = 50-100 |
| M2pep- zoledronic acid- polymer | 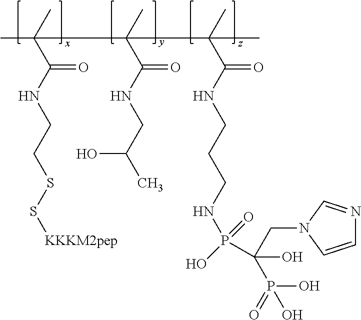 |
| | X = 5-10 |
| | Y = 100-400 |
| | Z = 50-200 |
| M2pep-CpG- polymer | 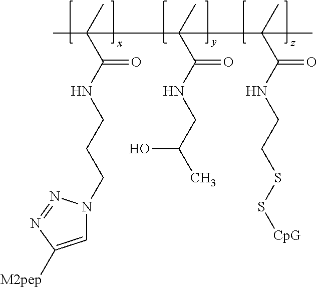 |
| | X = 5-10 |
| | Y = 100-400 |
| | Z = 5-20 |
| M2pep- | [M2pep]ₙ-Diphtheria protein sequence |
| diphtheria toxin | n = 1-5 |

In certain embodiments, the invention provides an M2 macrophage-binding agent comprising any of the peptides disclosed herein. Such agents may be useful for improved targeting of the peptides. In one non-limiting example, an M2 peptide could be incorporated in a loop region of a protein for targeting. In another example, the peptide could be cloned into a chimeric antigen receptor. A further example includes cloning the peptide into a virus loop to retarget virus.

Those skilled in the art will appreciate that certain variants thereof will be useful in the methods of the invention. A peptide “variant,” as used herein, is a peptide that differs from the peptides described herein in one or more substitutions, deletions, additions and/or insertions, such that the M2 macrophage-binding activity of the peptide is not substantially diminished. In other words, the ability of a variant to modulate M2 Macrophage binding may be enhanced or unchanged, relative to the M2 Macrophage-binding peptides specifically described herein, or may be diminished by less than 50%, and preferably less than 20%, relative to the M2 Macrophage-binding peptides specifically described herein. Such variants may generally be identified by modifying one of the above peptide sequences and evaluating the activity of the modified peptide using assays as described herein. Peptide variants preferably exhibit at least about 85%, more preferably at least about 90% and most preferably at least about 95% identity (determined as described above) to the identified peptides.

Preferably, a variant contains conservative substitutions. A “conservative substitution” is one in which an amino acid is substituted for another amino acid that has similar properties, such that one skilled in the art of peptide chemistry would expect the secondary structure and hydropathic nature of the peptide to be substantially unchanged. Amino acid substitutions may generally be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Other groups of amino acids that may represent conservative changes include: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his. A variant may also, or alternatively, contain nonconservative changes. In a preferred embodiment, variant peptides differ from a native sequence by substitution, deletion or addition of five amino acids or fewer.

In another aspect, the present invention provides isolated nucleic acids encoding the peptide of any aspect or embodiment of the invention. The isolated nucleic acid sequence may comprise RNA or DNA. As used herein, “isolated nucleic acids” are those that have been removed from their normal surrounding nucleic acid sequences in the genome or in cDNA sequences. Such isolated nucleic acid sequences may comprise additional sequences useful for promoting expression and/or purification of the encoded protein, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the polypeptides of the invention.

In a further embodiment, the present invention provides nucleic acid expression vectors comprising the isolated nucleic acid of any embodiment of the invention operatively linked to a suitable control sequence. “Recombinant expression vector” includes vectors that operatively link a nucleic acid coding region or gene to any control sequences capable of effecting expression of the gene product. “Control sequences” operably linked to the nucleic acid sequences of the invention are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors can be of any type known in the art, including but not limited plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive). The construction of expression vectors for use in transfecting prokaryotic cells is also well known in the art, and thus can be accomplished via standard techniques. (See, for example, Sambrook, Fritsch, and Maniatis, in: Molecular Cloning, A Laboratory Manual, Cold Spring Harbor Laboratory Press, 1989; Gene Transfer and Expression Protocols, pp. 109-128, ed. E. J. Murray, The Humana Press Inc., Clifton, N.J.), and the Ambion 1998 Catalog (Ambion, Austin, Tex.). The expression vector must be replicable in the host organisms either as an episome or by integration into host chromosomal DNA. In a preferred embodiment, the expression vector comprises a plasmid. However, the invention is intended to include other expression vectors that serve equivalent functions, such as viral vectors.

In another embodiment, the present invention provides recombinant host cells comprising the nucleic acid expression vectors of the invention. The host cells can be either prokaryotic or eukaryotic. The cells can be transiently or stably transfected or transduced. Such transfection and transduction of expression vectors into prokaryotic and eukaryotic cells can be accomplished via any technique known in the art, including but not limited to standard bacterial transformations, calcium phosphate co-precipitation, electroporation, or liposome mediated-, DEAE dextran mediated-, polycationic mediated-, or viral mediated transfection. (See, for example, Molecular Cloning: A Laboratory Manual (Sambrook, et al., 1989, Cold Spring Harbor Laboratory Press; Culture of Animal Cells: A Manual of Basic Technique, 2^{nd} Ed. (R. I. Freshney. 1987. Liss, Inc. New York, N.Y.). A method of producing a polypeptide according to the invention is an additional part of the invention. The method comprises the steps of (a) culturing a host according to this aspect of the invention under conditions conducive to the expression of the polypeptide, and (b) optionally, recovering the expressed polypeptide. The expressed polypeptide can be recovered from the cell free extract, cell pellet, or recovered from the culture medium. Methods to purify recombinantly expressed polypeptides are well known to the man skilled in the art.

In a further aspect, the present invention provides pharmaceutical compositions, comprising a peptide, nucleic acid, nucleic acid expression vector, biocompatible polymer, and/or recombinant host cell of any aspect or embodiment of the invention, and a pharmaceutically acceptable carrier. The pharmaceutical compositions of the invention can be used, for example, in the methods of the invention described below. The pharmaceutical composition may comprise in addition to the polypeptides, nucleic acids, etc. of the invention (a) a lyoprotectant; (b) a surfactant; (c) a bulking agent; (d) a tonicity adjusting agent; (e) a stabilizer; (f) a preservative and/or (g) a buffer.

In some embodiments, the buffer in the pharmaceutical composition is a Tris buffer, a histidine buffer, a phosphate buffer, a citrate buffer or an acetate buffer. The pharmaceutical composition may also include a lyoprotectant, e.g. sucrose, sorbitol or trehalose. In certain embodiments, the pharmaceutical composition includes a preservative e.g. benzalkonium chloride, benzethonium, chlorohexidine, phenol, m-cresol, benzyl alcohol, methylparaben, propylparaben, chlorobutanol, o-cresol, p-cresol, chlorocresol, phenylmercuric nitrate, thimerosal, benzoic acid, and various mixtures thereof. In other embodiments, the pharmaceutical composition includes a bulking agent, like glycine. In yet other embodiments, the pharmaceutical composition includes a surfactant e.g., polysorbate-20, polysorbate-40, polysorbate-60, polysorbate-65, polysorbate-80 polysorbate-85, poloxamer-188, sorbitan monolaurate, sorbitan monopalmitate, sorbitan monostearate, sorbitan monooleate, sorbitan trilaurate, sorbitan tristearate, sorbitan trioleaste, or a combination thereof. The pharmaceutical composition may also include a tonicity adjusting agent, e.g., a compound that renders the formulation substantially isotonic or isoosmotic with human blood. Exemplary tonicity adjusting agents include sucrose, sorbitol, glycine, methionine, mannitol, dextrose, inositol, sodium chloride, arginine and arginine hydrochloride. In other embodiments, the pharmaceutical composition additionally includes a stabilizer, e.g., a molecule which, when combined with a protein of interest substantially prevents or reduces chemical and/or physical instability of the protein of interest in lyophilized or liquid form. Exemplary stabilizers include sucrose, sorbitol, glycine, inositol, sodium chloride, methionine, arginine, and arginine hydrochloride.

The peptides, nucleic acids, etc. of the invention may be the sole active agent in the pharmaceutical composition, or the composition may further comprise one or more other active agents suitable for an intended use.

The peptides, nucleic acids, nucleic acid expression vectors, biocompatible polymers, and/or the recombinant host cells of the present invention can be present in the pharmaceutical composition at about 0.1 mg/L, 0.25 mg/L, 0.5 mg/L, 0.75 mg/L, 1.0 mg/L, 2.0 mg/L, 5.0 mg/L, 7.5 mg/L, 10 mg/L, 25 mg/L, 50 mg/L, or more.

The pharmaceutical compositions described herein generally comprise a combination of a compound described herein and a pharmaceutically acceptable carrier, diluent, or excipient. Such compositions are substantially free of non-pharmaceutically acceptable components, i.e., contain amounts of non-pharmaceutically acceptable components lower than permitted by US regulatory requirements at the time of filing this application. In some embodiments of this aspect, if the compound is dissolved or suspended in water, the composition further optionally comprises an additional pharmaceutically acceptable carrier, diluent, or excipient. In other embodiments, the pharmaceutical compositions described herein are solid pharmaceutical compositions (e.g., tablet, capsules, etc.).

The compositions described herein could also be provided as a dietary supplement as described by the US regulatory agencies.

These compositions can be prepared in a manner well known in the pharmaceutical art, and can be administered by any suitable route. In a preferred embodiment, the pharmaceutical compositions and formulations are designed for oral administration. Conventional pharmaceutical carriers, aqueous, powder or oily bases, thickeners and the like may be necessary or desirable.

The pharmaceutical compositions can be in any suitable form, including but not limited to tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

In another aspect, the present invention provides a polymer comprising two or more repeating units that form a backbone, wherein the repeating units comprise at least one unit comprising a conjugated M2 macrophage-binding peptide and optionally one or more unconjugated units. The unconjugated repeating units may have side chains containing but not limited to carboxybetaine, sulfobetaine, hydroxyls, and amines. The polymer may comprise repeating units that form a backbone wherein at least 2 up to 100 repeat units or up to 200 repeat units have a conjugated M2 macrophage-binding peptides. The M2peps useful in this aspect can be any M2peps of the present invention. In certain embodiments, the plurality of conjugated M2peps includes M2peps with sequences different from one another. In certain embodiments, each of the plurality of pendant M2peps have the same sequence.

In certain embodiments, the polymer further comprises a therapeutic molecule coupled to the polymer. In certain embodiments, the therapeutic molecule is selected from the group consisting of small molecules, peptides, and nucleic acids. In certain embodiments, the small molecule is a bisphosphonate derivative. In certain embodiments, the small molecule is an anticancer agent. In certain embodiments, the therapeutic agent is a peptide, wherein the peptide is a pro-apoptotic or a pro-inflammatory peptide. In certain embodiments, the therapeutic agent is a nucleic acid molecule, wherein the nucleic acid molecule is an siRNA.

In one embodiment, the polymer is water-soluble at concentrations >10 mg/L.

Polymers, as described herein, may be of any length between about 2 to about 500 repeating units in length, or more typically, between 10 and 300 units in length. Preferably, the polymer is produced by controlled polymerization. The polymer typically has a molecular weight of 5-60 kDa, but can be between about 3 and about 300 kDa depending on the repeating units employed. The polymer typically has a degree of polymerization in the range from 20-400, but it can be between about 5 and about 600 depending on the repeating units employed. In one embodiment, the polymer is a linear polymer. Other embodiments of the polymer include cyclic and branched polymer conformations, including star and sunflower conformations. In one embodiment, the polymer is synthesized by living polymerization techniques such as reversible addition-fragmentation chain transfer (RAFT) polymerization or Atom transfer radical polymerization (ATRP). Other methods of polymer synthesis that are known in the art may be employed.

The invention provides polymers comprising repeating units that form a backbone, wherein a fraction of the repeating units comprises a conjugated M2pep peptide. In certain embodiments, the ratio of repeating units containing a conjugated M2pep peptide to unconjugated units is 99:1, 98:2, 97:3, 96:4, 95:5, 90:10; 85:15, 80:20, 75:25, 70:30, 60:40, 50:50, 40:60, 30:70, 25:75, 80:20, 15:85, 10:90, 5:95, 4:96, 3:97, 2:98, and 1:99. In certain embodiments, the ratio of conjugated repeating units to unconjugated units is 95:5, 90:10 or 80:20. In certain embodiments the conjugated units are present at a ratio of 4 unconjugated units to each conjugated unit.

The invention provides a polymer to which a plurality of M2peps are bound. In one embodiment, one of the repeating units is comprised of polymerized (hydroxyethyl) methacrylate (HEMA). In other embodiments, the polymer comprises a zwitterion (e.g., polycarboxybetaine, polysulfobetaine, and polyphosphobetaine).

Examples of repeating units suitable for use in synthesis of the polymer described herein include N-(3-aminopropyl)methacrylamide (APMA), N, N-diethylacrylamide, N-[3-(Dimethylamino)propyl]methacrylamide, N-hydroxyethyl acrylamide, 2-aminoethyl methacrylate, 2-(Dimethylamino)ethyl methacrylate, 2-ethoxyethyl methacrylate, Ethylene glycol methyl ether methacrylate, Ethyl methacrylate, Glycidyl methacrylate, Glycosyloxyethyl methacrylate and other carbohydrate methacrylates, acrylates, methacrylamides, or methacrylates; 2-hydroxyethyl methacrylate, Hydroxypropyl methacrylate, Poly(ethylene glycol) methacrylate, Propyl methacrylate, 3-sulfopropyl methacrylate, Triethylene glycol methyl ether methacrylate, 2-(diethylamino)ethyl acrylate, 2-(dimethylamino)propyl acrylate, Di(ethylene glycol)2-ethylhexyl ether acrylate, Ethyl-2-ethylacrylate, 2-hydroxyethyl acrylate, Poly(ethylene glycol) methyl ether acrylate, 24-hydroxybutlyl acrylate, and Aminoacid-N-carboxyanhydride monomers. Additional examples of hydrophilic monomers include N-acryloylamido-ethoxyethanol, N-isopropylacrylamide, N-isopropylmethacrylamide, methacrylamide, 2-acrylamido-2-methyl-1-propanesulfonic acid, N-Tris(hydroxymethyl)methyl]acrylamide, Methyl methacrylate, 2-N-morpholinoethyl methacrylate, 2-(2-oxo-1-imidazolidinyl)ethyl methacrylate, pyridyl disulfide methacrylamide, glycidyl methacrylate, 2-hydroxyethyl methacrylate, methacrylic acid N-hydroxysuccinimide, mono-2-(methacryloyloxy)ethyl maleate, 2-Carboxyethyl acrylate, propargyl acrylate (or protected form), acrylate-PEG-maleimide, amino acid-N-carboxyanhydride monomers, alkyne monomers, maleimide monomers, and 3-sulfopropyl acrylate.

The M2peps can be incorporated to the polymer via conjugation to reactive groups on the polymer. For example, the M2peps can be conjugated to N-hydroxysuccinimidyl (NHS) reactive groups on the polymer via the ε-amine on the C-terminus lysine. Other examples of conjugation chemistry that can be employed for conjugation of M2peps to the polymer include, but are not limited to, azide-alkyne coupling chemistry, thiol-ene chemistries, thiol-disulfide exchange, nucleophilic substitution reactions, and hydrazone bond chemistry. The conjugation provides for multivalent display of M2peps that are pendant at multiple intervals along the length of the polymer.

In certain embodiments, the unconjugated repeating units comprise a polycarboxybetaine, polysulfobetaine, polyphosphobetaine, (hydroxyethyl)methacrylate (HEMA), or poly(N-(2-hydroxypropyl)methacrylamide) (HPMA). In certain embodiments, at least one conjugated unit is conjugated N-hydroxysuccinimidyl ester methacrylate (NHSMA), pyridyl disulfide methacrylamide (PDSMA), N-(3-aminopropyl) methacrylamide hydrochloride (APMA), poly(propylacrylic acid) (PPAA), glycidyl methacrylate, 2-hydroxyethyl methacrylate, methacrylic acid N-hydroxysuccinimide, mono-2-(methacryloyloxy)ethyl maleate, 2-Carboxyethyl acrylate, propargyl acrylate, acrylate-PEG-maleimide, or amino acid-N-carboxyanhydride monomer. In certain embodiments, the conjugated units are conjugated N-NHSMA, and wherein the NHSMA is present as a co-polymer with HEMA (p(HEMA-co-NHSMA)).

In addition to M2peps, other agents can be bound to the polymer. Examples of agents that can be bound to the polymer include therapeutic agents as discussed above and imaging agents. Representative examples of imaging agents include, but are not limited to, MR contrast agents (e.g., gadolinium), positron emission tomography (PET) contrast agents, or fluorophores. Representative examples of therapeutic agents include chemotherapy or immunotherapy drugs, pro-apoptotic peptides or pro-inflammatory peptides.

In certain embodiments, the polymer has the structure:

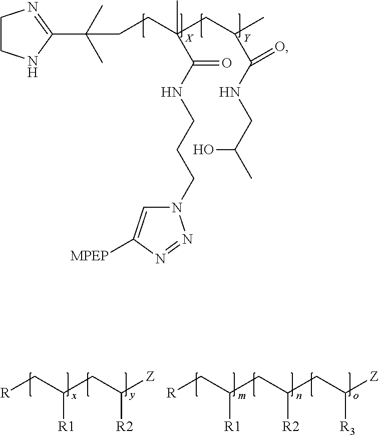

wherein
R1 is the M2 macrophage-binding peptide,
MPEP is the macrophage-binding peptide,
R2 is a therapeutic or imaging cargo,
X is between 2-500, or between 4-1000; and
Y is between 0-500, or 4-1000 and X and Y can either be in block segments for block copolymers or intermixed for random or statistical copolymers.

In another aspect, the present invention provides a method of treating or ameliorating a subject suffering from cancer comprising: administering a therapeutically effective dose of an isolated peptide according to any embodiment of the invention or a biocompatible polymer according to any embodiment of the invention, wherein the isolated M2 macrophage-binding peptide or biocompatible polymer comprise a therapeutic agent, thereby delivering the therapeutic agent and treating or ameliorating the cancer.

Treatment includes prophylaxis and therapy. Prophylaxis or therapy can be accomplished by a single direct injection at a single time point or multiple time points to a single or multiple sites. Administration can also be nearly simultaneous to multiple sites. Patients or subjects include mammals, such as human, bovine, equine, canine, feline, porcine, and ovine animals. The subject is preferably a human.

The compositions are administered in any suitable manner, optionally as pharmaceutically acceptable salts or with pharmaceutically acceptable carriers. Suitable methods of administering compositions, moieties, and molecules in the context of the present invention to a subject are available, and, although more than one route can be used to administer a composition, a particular route can often provide a more immediate and more effective reaction than another route.

The dose administered to a patient, in the context of the present invention, should be sufficient to effect a beneficial therapeutic response in the patient over time, to delay onset of disease, or to inhibit disease progression. Thus, the composition is administered to a subject in an amount sufficient to alleviate, reduce, and cure or at least partially delay or arrest symptoms and/or complications from the disease. An amount adequate to accomplish this is defined as a “therapeutically effective dose.”

A suitable dose is an amount that, when administered as described herein, is capable of promoting a reduction in symptoms, and preferably at least 10-50% improvement over the basal (i.e., untreated) level. Such therapies should lead to an improved clinical outcome (e.g., more frequent remissions, complete or partial or longer disease-free survival) in patients as compared to untreated patients. In general, for pharmaceutical compositions comprising one or more peptides, the amount of each peptide present in a dose ranges from about 100 μg to 5 mg per kg of host. Suitable volumes will vary with the size of the patient, but will typically range from about 0.1 mL to about 5 mL.

Routes and frequency of administration of the therapeutic compositions disclosed herein, as well as dosage, will vary from individual to individual, and may be readily established using standard techniques. In general, the pharmaceutical compositions may be administered, by injection or implantation (e.g., intracutaneous, intratumoral, intramuscular, intraperitoneal, intravenous, intrathecal, epidural or subcutaneous), intranasally (e.g., by aspiration) or orally. Typically the administration is intravenous. In one example, between 1 and 10 doses may be administered over a 52 week period. Preferably, 6 doses are administered, at intervals of 1 month, and booster administrations may be given periodically thereafter, as indicated. Alternate protocols may be appropriate for individual patients. In one embodiment, 2 intradermal injections of the composition are administered 10 days apart. In another embodiment, a dose is administered daily or once every 2 or 3 days over an extended period, such as weeks or months.

In general, an appropriate dosage and treatment regimen provides the active compound(s) in an amount sufficient to provide therapeutic and/or prophylactic benefit. Such a response can be monitored by establishing an improved clinical outcome (e.g., more frequent remissions, complete or partial, or longer disease-free survival) in treated patients as compared to non-treated patients.

### Examples

### Materials

Protected amino acids and 2-(6-chloro-1H-benzotriazole-1-yl)-1,1,3,3-tetramethylaminium hexafluorophosphate (HCTU) were purchased from AAPPTec (Louisvulle, Ky.) and AnaSpec (Fremont, Calif.). NovaPEG Rink Amide was purchased from Merck Millipore (Billerica, Mass.). Collagenase (C0130), dispase II, triisopropylsilane (TIPS), 1,2-ethanedithiol (EDT), 1,3-dimethoxybenzene (DMB), tris(2-carboxyethyl)phosphine hydrochloride (TCEP), copper(II) sulfate pentahydrate (CuSO₄.5H₂O), tris(3-hydroxypropyltriazolylmethyl)amine (THPTA), and sodium ascorbate were purchased from Sigma-Aldrich (St. Louis, Mo.). Sulfo-Cy5-alkyne was purchased from Lumiprobe (Hallandale Beach, Fla.). Streptavidin FITC was purchased from eBioscience (San Diego, Calif.). Pacific blue anti-mouse F4/80 antibody (Clone BM8) was purchased from Life Technologies (Grand Island, N.Y.). PerCP anti-mouse/human CD11b antibody (Clone M1/70) and anti-mouse CD16/32 antibody (Fc receptor block, clone 93) were purchased from BioLegend (San Diego, Calif.). FITC anti-mouse Ly-6G antibody (Clone 1A8) was purchased from BD Pharmingen (San Diego, Calif.). Mouse macrophage colony-stimulating factor (M-CSF), interleukin-4 (IL-4), and interferon-γ (IFN-γ) were purchased from R&D Systems (Minneapolis, Minn.). Lipopolysaccharide (LPS) was purchased from InvivoGen (San Diego, Calif.). Normal mouse serum (Catalog number 10410) and other reagents were purchased from Thermo Fisher Scientific (Waltham, Mass.).

Peptide synthesis was performed on an automated PS3 peptide synthesizer (Protein Technologies, Phoenix, Ariz.) following the standard Fmoc solid phase peptide synthesis chemistry. When needed, amino acids were manually coupled by incubation in a solution of amino acid and HCTU dissolved in 0.4 M N-methylmorpholine in DMF for 3 h. The coupling reaction was checked for completion by Kaiser Test as previously described [18]. Fmoc protecting groups were removed by two 30 min incubations in 20% (v/v) piperidine in DMF. Biotinylated peptides were synthesized from the biotinylated resin manually as follows: Fmoc-Lys(Mtt)-OH was first coupled to the NovaPEG rink amide resin. The Mtt-protecting group was removed by 3-min incubations in 1.8% (v/v) TFA in DCM until completion (15-20 times) as indicated by the disappearance of the yellow color (Mtt cation) in the drained deprotection solution. Biotin was then coupled to the resin, and the subsequent amino acid extension was continued on the peptide synthesizer. Peptides were acetylated at the N-terminus in acetic anhydride/triethylamine/DCM (1:1:5 v/v/v) for 2 h. Peptides were cleaved in TFA/TIPS/EDT/DMB (90:2.5:2.5:5 v/v/v/v) for 2.5 h. EDT was included in the cleavage solution only for the cysteine-containing peptides. The cleaved peptides were precipitated in cold ether twice and purified by RP-HPLC (Agilent 1200, Santa Clara, Calif.) using Phenomenex Fusion-RP C18 semi-preparative column (Torrance, Calif.) in H₂O (0.1% TFA) as a mobile phase A and ACN (0.1% TFA) as a mobile phase B. Disulfide cyclization of the purified peptides was performed by incubation in deaerated 0.1 M ammonium bicarbonate buffer (4 mg/mL peptide concentration) for 2 days. The peptides were then desalted using the HyperSep™ C18 cartridge and confirmed for purity with RP-HPLC. Molecular weights of the purified peptides were confirmed by matrix-assisted laser desorption/ionization time-of-flight mass spectrometry (MALDI-TOF MS, Bruker Daltonics, Billerica, Mass.).

Azide-functionalized peptides were synthesized following the procedure described above replacing Fmoc-Lys(Mtt)-OH with Fmoc-D-Lys(N₃)—OH at the C-terminus. Sulfo-Cy5 labeling, used for imaging or tracking the peptide, via copper(I)-catalyzed azide-alkyne cycloaddition (CuAAC) was performed by reacting 1 eq. of peptide with 1.3 eq. of sulfo-Cy5-alkyne in the presence of 5 eq. of CuSO₄.5H₂O, THPTA, and sodium ascorbate. CuSO₄.5H₂O and THPTA were pre-mixed for a few minutes before adding to the peptide solution. Sodium ascorbate was added last. The final peptide concentration was 1 mM in H₂O/DMF (1:1 v/v). The conjugation reaction was maintained at 37° C. for 1.5 h after which any solid precipitate was centrifuged. The supernatant was initially cleaned up by eluting through Sep-Pak C18 cartridge, concentrated by rotary evaporator, and then purified by RP-HPLC. Peptide was cyclized after sulfo-Cy5 labeling.

### Serum Stability Study

The peptide of interest (30 μL of 10 mg/mL stock solution in dH₂O) was added to normal mouse serum (300 μL) and incubated at 37° C. in an incubator. At each time point, an aliquot of 40 μL was drawn and an equal volume of ACN was added to the aliquot to precipitate serum proteins. The cloudy mixture was centrifuged at 10,000 rpm for 5 min and then supernatant removed. A solution of 1:1 H₂O/ACN (80 μL) was added to the pellet and sonicated for 10 min to further extract the remaining peptides. This resuspended mixture was then centrifuged, and the supernatant was pooled with the former and vacuum-dried on a Speedvac machine. The dried pellet was solubilized in H₂O (50 μL) by sonication for 10 min. The mixture was then centrifuged, and the supernatant was drawn and analyzed by MALDI-TOF MS.

### Bone Marrow Harvest

All animal handling protocols were approved by the University of Washington Institutional Animal Care and Use Committee. Bone marrow harvest was performed following the previously reported protocol [14]. Briefly, femur and tibia were excised from 6-8 week-old female c57bl6/027 mice. Cuts were made on the ends of each bone, and RPMI 1640 medium was used to flush out bone marrow cells via 18G needle. The cells were cultured on petri dishes in RPMI 1640 medium supplemented with 20% donor horse serum, 1% antibiotic-antimycotic (AbAm), and 20 ng/mL M-CSF. After 7 d of culture, macrophages were activated by replacing M-CSF with 25 ng/mL IFN-γ and 100 ng/mL LPS for M1 macrophage or 25 ng/mL IL-4 for M2 macrophage. The macrophages were activated for 2 d and then scraped off the petri dishes with cell lifter for binding studies.

### Binding Study

Peptide solutions at different concentrations for binding study were prepared by dilutions from the stock solution (10 mg/mL in dH₂O) with PBS containing 1% BSA (PBSA). M1 and M2 macrophages (50,000 cells/well) were seeded on a black 96-well plate and incubated in the peptide solutions on ice for 20 min. Unbound peptides were washed off with PBSA twice. The macrophages were subsequently incubated with streptavidin-FITC for 15 min on ice to probe for the bound peptides. Excess streptavidin-FITC was washed off with PBSA twice, and the macrophages were then resuspended in PBS for analysis by MACSQuant Flow Cytometer (Miltenyi Biotec, San Diego, Calif.). Propidium iodide was added to the samples before the data acquisition to discriminate dead cells. Flow cytometry data were analyzed with FlowJo Analysis Software (Tree Star, Ashland, Oreg.). Binding study of peptides post serum incubation was similarly performed with the following additions: The peptides (5 mM stock solution in H₂O) were diluted in serum to a final concentration of 0.5 mM and incubated at 37° C. in an incubator. At each time point, an aliquot of the peptides was drawn and diluted in PBS to the final concentration for the binding study. The diluted aliquot was heated at 80° C. for 30 min to inactivate serum proteins, stored in a −20° C. freezer, and thawed for use in the binding study after aliquots at all time points were collected.

Biotin and fluorescently labeled peptides were used for the purpose of assessing peptide binding.

### In Vivo Biodistribution Study in CT-26 and 4T1 Tumor Models

CT-26 colon carcinoma cells and 4T1 mammary carcinoma cells were maintained in DMEM medium supplemented with 10% fetal bovine serum (FBS) and 1% Penicillin Streptomycin. Subcutaneous tumors were formed by the injection of 10⁶ CT-26 or 4T1 cells into the right flank or mammary fat pad, respectively, of 6 week-old female BALB/c mice. Tumor-bearing mice were used 2 weeks post inoculation at a maximum tumor diameter of 1.5 cm. The mice were injected retro-orbitally with 150 μL of PBS control or sulfo-Cy5-labeled peptides (2.24 nmol in PBS with 10% DMSO). At 20 min after peptide injection, mice were anesthetized with a ketamine/xylazine cocktail and perfused with PBS. Organs were harvested and imaged on the Caliper Xenogen IVIS (PerkinElmer, Hopkinton, Mass.). The fluorescence intensity emitted from each organ was subsequently quantified utilizing Xenogen's Living Image software. Following imaging, each tumor was then processed into single cell suspension by mincing into small pieces, homogenizing on gentleMACS Dissociator (Miltenyi Biotec, San Diego, Calif.), and incubating in 5 mL RPMI 1640 medium with 100 μL collagenase (10,000 CDU/mL stock solution) and 100 μL dispase II (32 mg/mL stock solution) for 40 min in the TC incubator. The dissociated single cells were collected through 70 μm cell strainer and plated on a black round bottom 96-well plate at the density of 200,000 cells/well. Incubation of the cells with Fc receptor block was performed prior to staining with CD11b, Ly-6G, and F4/80 antibodies. Finally, the cells were fixed with 4% paraformaldehyde and analyzed with flow cytometer.

### Statistical Analysis

Normalized median fluorescent intensity values in the binding studies were presented as mean±SD of triplicate in the same experiment. Statistical significance in binding between M1 and M2 macrophages or binding between M2pep and its modified analogs was evaluated by Student's unpaired t test. For clarity of presentation, only statistical significance at 50 μM concentration was labeled to elucidate the general trend in binding activity. Analysis of the binding of M2pep analogs post-serum incubation at the 24-h time point was performed by One-way ANOVA with Tukey's post-hoc tests. Accumulation of M2pep analogs in different organs were compared by Student's unpaired t-test. Finally, intratumoral biodistribution of each M2pep analog in different tumor subpopulations as well as comparison of accumulation in M2-like TAMs of the M2pep analogs were analyzed by one-way ANOVA with Tukey's post-hoc tests. All data analysis was processed on GraphPad Prism 6 (GraphPad Software Inc., La Jolla, Calif.). P <0.5 was considered as statistically significant.

### Results and Discussion

TABLE 1
Amino acid sequences of exemplary M2pep analogs

| | |
|---|---|
| Name | Sequence |

Linear M2pep analogs

| | |
|---|---|
| M2pep | YEQDPWGVKWWYGGGSKK (SEQ ID NO: 44) |
| AcM2pep | Ac-YEQDPWGVKWWYGGGSKKK (SEQ ID NO: 45) |
| W10w | Ac-YEQDPWGVKwWYGGGSKKK (SEQ ID NO: 46) |
| W(10,11)w | Ac-YEQDPWGVKwwYGGGSKKK (SEQ ID NO: 47) |
| W10P | Ac-YEQDPWGVKPWYGGGSkkk (SEQ ID NO: 48) |
| W10Y | Ac-YEQDPWGVKYWYGGGSkkk (SEQ ID NO: 49) |
| K9R | Ac-YEQDPWGVRWWYGGGSKKK (SEQ ID NO: 50) |
| AcM2pep(RY) | Ac-YEQDPWGVRYWYGGGSkkk (SEQ ID NO: 51) |
| W10(P,D,T,R,H) | |
| (Crude mixture) | Ac-YEQDPWGVK(P,D,T,R,H)WYGGGSkkk (SEQ ID NO: 52) |
| Y12y | Ac-YEQDPWGVRYWyGGGSkkk (SEQ ID NO: 53) |
| P5Hyp | Ac-YEQD(Hyp)WGVRYWyGGGSkkk (SEQ ID NO: 54) |
| R0 | Ac-RYEQDPWGVRYWyGGGSkkk (SEQ ID NO: 55) |

Cyclized M2pep analogs

| | |
|---|---|
| Disulfide-cyclized M2pep(RY) | 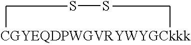 |
| | (SEQ ID NO: 16) |
| cRYWY | 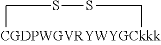 |
| | (SEQ ID NO: 17) |
| cRYW | 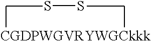 |
| | (SEQ ID NO: 18) |
| Amide-cyclized M2pep(RY) | 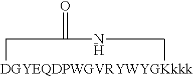 |
| | (SEQ ID NO: 19) |
| Triazole-cyclized M2pep(RY) | 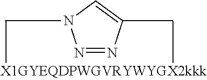 |
| | (SEQ ID NO: 20) |
| DFBP-cyclized M2pep(RY) | 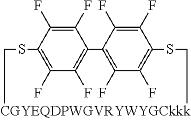 |
| | (SEQ ID NO: 21) |
| DFS-cyclized M2pep(RY) | 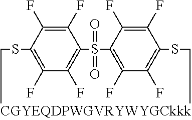 |
| | (SEQ ID NO: 22) |
| DFBP-cyclized M2pep | 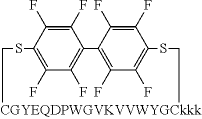 |
| | (SEQ ID NO: 23) |
| FF-cyclized M2pep(RY) | 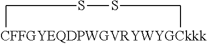 |
| | (SEQ ID NO: 24) |
| F(F5)F(F5)-cyclized M2pep(RY) | 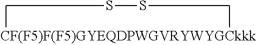 |
| | (SEQ ID NO: 25) |

Peptide sequences are written from N-terminus to C-terminus.
All peptides are amidated at the C terminus.
Small (lowercase) letters denote D-amino acids.
Amino acid residues involved in cyclization are in red.
X₁ = azidolysine
X₂ = propargylglycine
F(F5) = pentafluorophenylalanine

### The Effect of N-Terminal Acetylation on Serum Stability and Binding Activity of M2pepBiotin

In vivo applications of peptides as therapeutic or imaging agents are usually limited by their short serum half-life, typically on the order of a few minutes, due to degradation by serum proteases as well as clearance via kidneys and liver [19-21]. In regard to serum stability, several strategies including N-terminal acetylation, C-terminal amidation, D-amino acid substitution, and peptide stapling/macrocyclization have been utilized to impart peptide resistance against peptidase degradation [19,22]. In this study, serum stability of M2pep was first evaluated to determine peptidase-susceptible sites. A series of rationally-engineered peptides were then synthesized and evaluated for their serum stability and binding activity (Table 1).

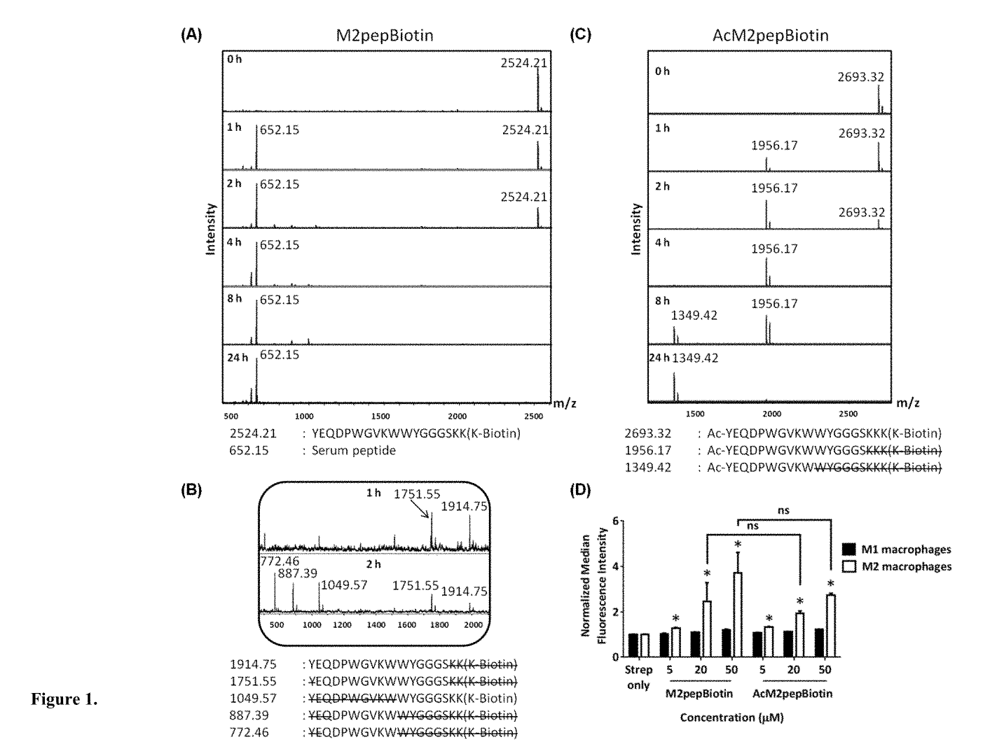

To evaluate serum stability of M2pep, the peptide was incubated in mouse serum, and aliquots of the serum at various time points were withdrawn for analysis of degradation patterns by MALDI-TOF MS. M2pepBiotin was quickly degraded in mouse serum and was no longer detected after 4 h of serum incubation (FIG. 1A). Instead, a serum peptide (652.15 Da) became the predominant signal detected from this time point onwards. Analysis of the degraded peptide fragments revealed that the peptide degradation is both exolytic from the N-terminus and endolytic within the peptide (FIG. 1B). Poor serum stability of this peptide therefore warranted investigation into more serum-resistant M2pep analogs.

N-terminal acetylation of peptides is a common peptide modification strategy shown to effectively prevent N-terminal peptide degradation by exopeptidases [23,24]. Hence, M2pep was acetylated at the N-terminus by reaction with acetic anhydride before cleavage from resin. The acetylated peptide, AcM2pepBiotin, was indeed resistant to N-terminal degradation (FIG. 1C). However, this modification did not improve the overall serum stability of the peptide since the peptide remained susceptible to endolytic degradation. With the N-terminal protection, the peptide degradation patterns became clearer showing two distinct peptide fragments corresponding to endolytic cleavages at W10/W11 and S16/K17 sites with the latter occurring with relatively faster kinetics.

Next, the binding specificity of AcM2pepBiotin to M2 versus M1 macrophage was evaluated using primary bone marrow-derived macrophages polarized to the M1 or M2 phenotypes as we previously described [14]. AcM2pepBiotin retained binding selectivity to M2 macrophages over M1 macrophages although with slight, but not statistically significant, reduction in binding activity compared to M2pep at the concentrations tested (FIG. 1D). Hence, we decided to acetylate all our subsequent peptides to protect against exolytic degradation and focused our next modifications at the W10/W11 and S16/K17 endolytic cleavage sites.

The effect of D-amino acid substitutions on serum stability and binding activity of AcM2pepBiotin

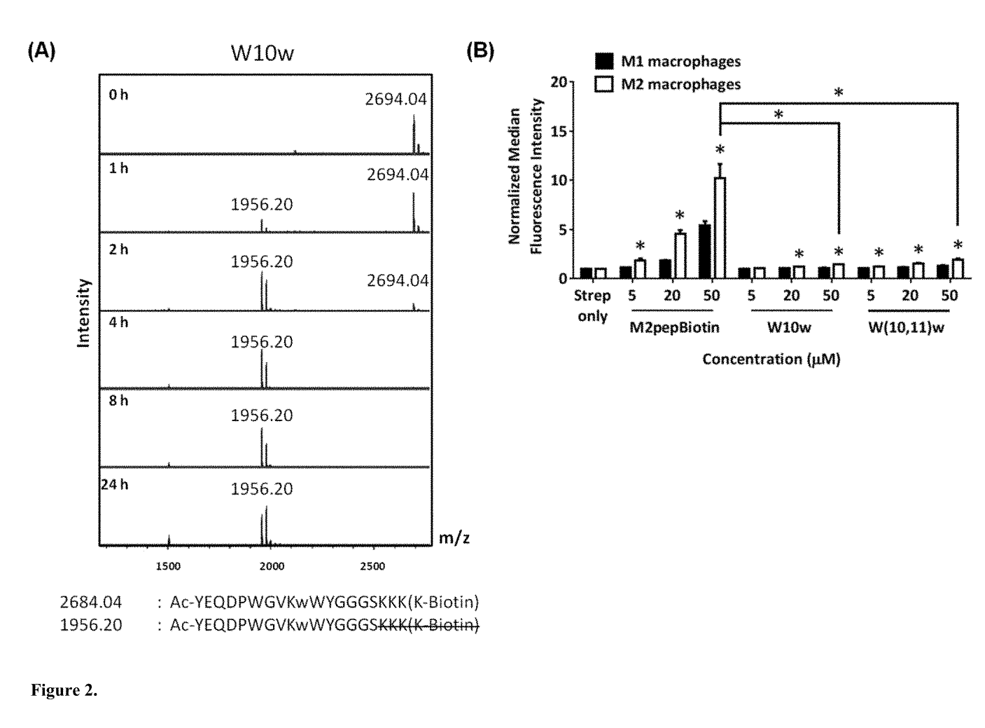

As reported previously, our high throughput sequencing of phage that bound to M2 macrophages showed a consensus motif of DPWXXXXW where X denoted other amino acids with less or no consensus [25]. Hence, we deduced that the motif may be essential for binding to the M2pep receptor and targeted our next peptide modification to the W10 position which lies in the non-consensus region. Since D-amino acids are not normally present in nature and substitution with D-amino acids into peptide sequences often confer serum stability [11], we explored the effect of D-tryptophan substitution at the W10 position (W10w). Despite its effective protection against endolytic cleavage at the W10/W11 site (FIG. 2A), the W10w substitution abrogated binding activity of the peptide (FIG. 2B). In addition, D-tryptophan substitutions to both W10 and W11 (W(10,11)w) also did not restore the binding activity of M2pep (data not shown).

### The Effect of L-Amino Acid Substitutions on Serum Stability and Binding Activity of AcM2pepBiotin

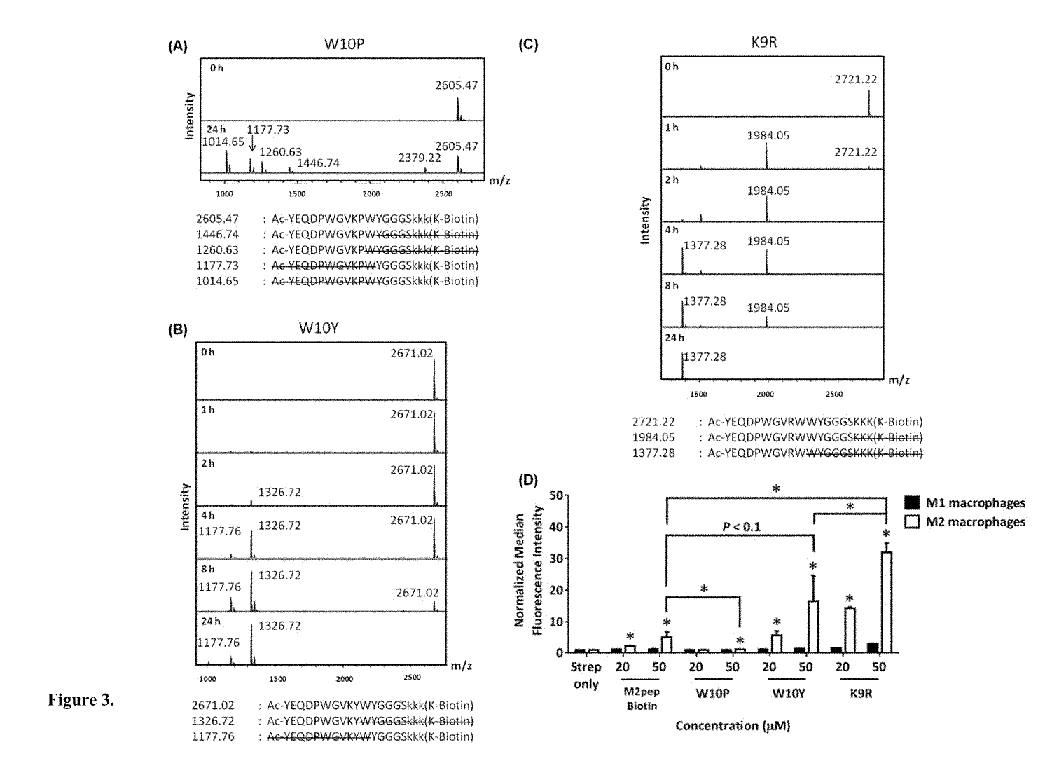

Next, we investigated how substitutions of W10 with other L-amino acids may help protect against degradation at the W10/W11 site. We first synthesized a W10P analog due to the apparent serum stability at the P5W6 site in AcM2pepBiotin and also a W10Y analog since tyrosine, like tryptophan, is an amino acid with a bulky aromatic side chain [26]. For both analogs, the trilysine spacer was also replaced with D-lysines to evaluate if this replacement would improve serum stability at the S16/K17 site. In addition, we also studied a K9R analog. K9 is the only lysine in the targeting region and being able to replace this lysine with arginine would allow us to synthesize AcM2pep with C-terminal lysine for facile and site-selective conjugation to drug cargos with activated NHS ester. D-lysine substitution at the trilysine spacer improved serum stability at S16/K17 site while W10P, W10Y, and K9R substitutions did not confer protection at the W10/W11 site (FIG. 3A). Interestingly, binding studies showed that both K9 and W10 amino acid positions are essential for binding activity of AcM2pepBiotin (FIG. 3B). In this case, K9R and W10Y substitutions significantly increased binding activity of AcM2pepBiotin while a W10P substitution abrogated the binding activity. Combining K9R and W10Y substitutions together (AcM2pep(RY)Biotin) did not improve the binding activity any further compared to the K9R analog and also did not improve serum stability at the W10/W11 site (Fig. S1). Given high natural abundance of pi-cation interactions between Lys/Arg and Phe/Tyr/Trp in many proteins [27], K9 may be involved in the interaction with the aromatic amino acids either on the M2pep itself or on the target receptor, and the higher binding affinity of the K9R analog may thus be attributed to the stronger pi-cation interaction of the relatively more hydrophobic arginine with the corresponding aromatic amino acid [28,29]. Additional investigation into W10(D,R,H) substitutions also did not give a positive result in term of improving serum stability (Fig. S2).

### The Effect of Cyclization on Serum Stability and Binding Activity of M2pepBiotin

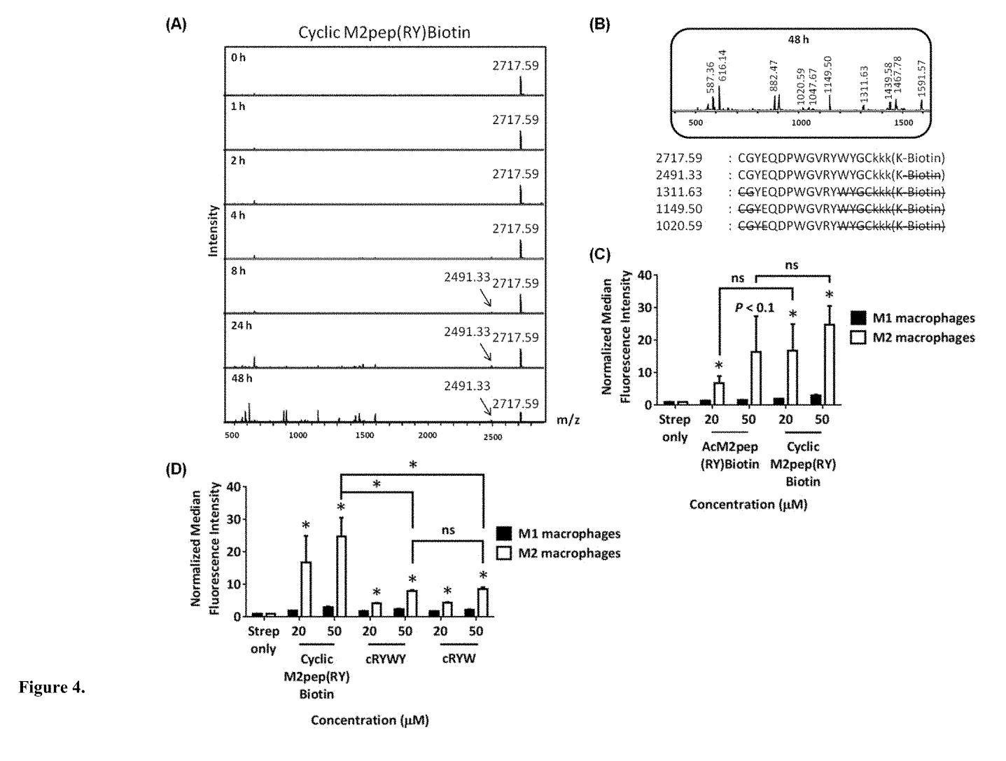

Head-to-tail cyclization of peptides may impose structural rigidity to the peptides and in several cases, leads to improvement in bioactivity and serum stability [30]. Hence, we modified linear M2pep(RY)Biotin with two flanking cysteines to enable disulfide cyclization of the peptide. The synthesized peptide (cyclic M2pep(RY)Biotin) had markedly improved serum stability over the linear AcM2pep(RY)Biotin (FIG. 4A). Unlike the linear, acetylated M2pep derivatives which were degraded within 2 to 8 hrs post-serum incubation, cyclic M2pep(RY)Biotin remained detectable by MALDI-TOF MS even after serum incubation for 48 h. No endolytic cleavage was observed at the W10/W11 site in cyclic M2pep(RY)Biotin for up to at least 8 h. At 24 and 48 h, however, signals corresponding to N-terminal degradation and W10/W11 endolytic cleavage were observed. This degradation might be attributed to reduction of the disulfide bond over time, which converts the peptide to the protease-accessible linear form (FIG. 4B). Interestingly, cleavage of biotin from the peptide was also observed at later time points and was not appreciated previously due to poor serum stability of the other analogs. Furthermore, cyclic M2pep(RY)Biotin has comparable binding activity to the linear AcM2pep(RY)Biotin, the highest affinity linear M2pep analog (FIG. 4C). Hence, in this study, we showed that cyclization of M2pep(RY)Biotin significantly imparted serum stability over all linear AcM2pep analogs while retaining high binding activity comparable to AcM2pep(RY)Biotin. In the future, more stable cyclization strategies, such as incorporation of azide- and alkyne-functionalized amino acids for cyclization via CuAAC, may be investigated to further improve serum stability of this peptide.

In an effort to truncate the cyclic M2pep(RY)Biotin sequence, we investigated the effect of deleting amino acids that lie outside the DPWXXXXW motif by synthesizing two cyclic M2pep(RY)Biotin analogs; 1) cRYWY having deletion of Y1, E2, and Q3 and 2) cRYW containing deletions of Y1, E2, Q3, and also Y12. Both cRYWY and cRYW analogs have significantly lower binding activity compared to cyclic M2pep(RY)Biotin implying that Y1, E2, and Q3 may be important for binding to M2 macrophages (FIG. 4D). However, Y12 may not be a critical residue since cRYWY and cRYW analogs have similar binding activity.

### The Effect of Y12y on Serum Stability and Binding Activity of AcM2pep(RY)Biotin

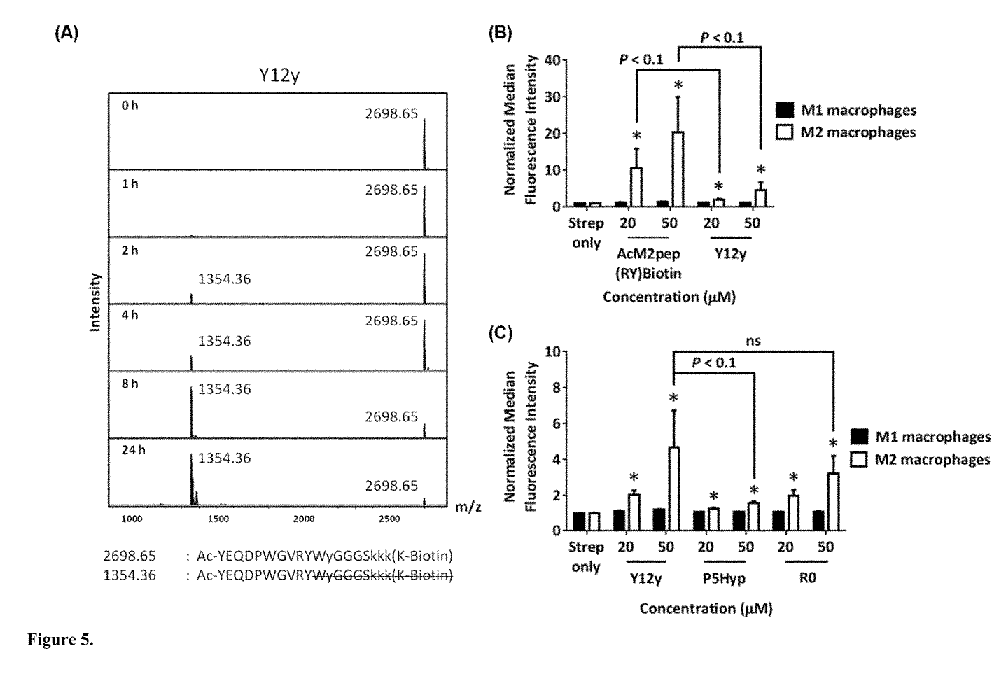

Since Y12 is not required for binding activity, we next synthesized linear AcM2pep(RY)Biotin with a D-tyrosine substitution at the Y12 position (Y12y) to evaluate if this substitution confers protection to the nearby W10/W11 cleavage site. However, the Y12y analog did not improve serum stability at the W10/W11 site (FIG. 5A) and also unexpectedly reduced binding activity of the peptide (FIG. 5B). Thus, while deletion of Y12 does not affect binding, substitution with a D-amino acid reduces binding affinity. Since AcM2pepBiotin shows trends of slightly reduced binding compared with M2pepBiotin (FIG. 1D), we investigated the effect of introducing an arginine (R0) at the start of the AcM2pep(RY)Biotin sequence to offset for loss in amine from acetylation of M2pep. In addition, we also substituted P5 with hydroxyproline (HyP), which has been shown previously to improve binding activity of another proline-containing peptide [31]. Both analogs were synthesized with the Y12y substitution, and binding study was evaluated in comparison to the Y12y analog. The R0 analog did not improve binding activity any further while P5Hyp significantly ablated the binding activity (FIG. 5C).

### Binding of M2pep Analogs Post Serum Incubation

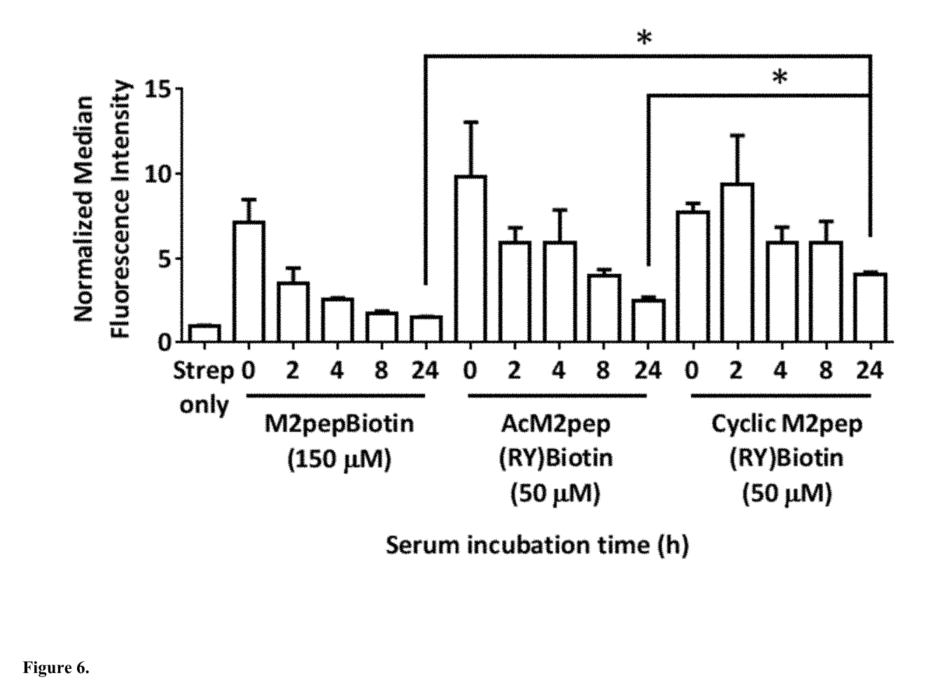

For TAM targeting applications, M2pep analogs will be exposed to serum during systemic circulation. Therefore, we next studied how the serum stability of M2pep analogs affects subsequent binding to target cells. Peptides were incubated with mouse serum at 37° C. to mimic peptidase exposure in systemic circulation, and aliquots of the serum were withdrawn at various time points for binding studies with polarized macrophages. In general, binding activity of the M2pep analogs corresponded with the peptides' serum stability (FIG. 6). Binding studies were performed at 150 μM of M2pepBiotin and 50 μM of AcM2pep(RY)Biotin and cyclic M2pep(RY)Biotin such that all the analogs had similar extent of binding at 0 h. M2pepBiotin had the fastest and greatest decline in binding activity while cyclic M2pep(RY)Biotin had the least decline which was also partially accounted by the cleavage of biotin. At 24 h, cyclic M2pep(RY)Biotin retained the highest binding activity compared to M2pepBiotin and AcM2pep(RY)Biotin reaffirming the highest serum stability of this M2pep analog observed in the previous study.

### Biodistribution of M2pep Analogs in CT-26 and 4T1 Tumor Models

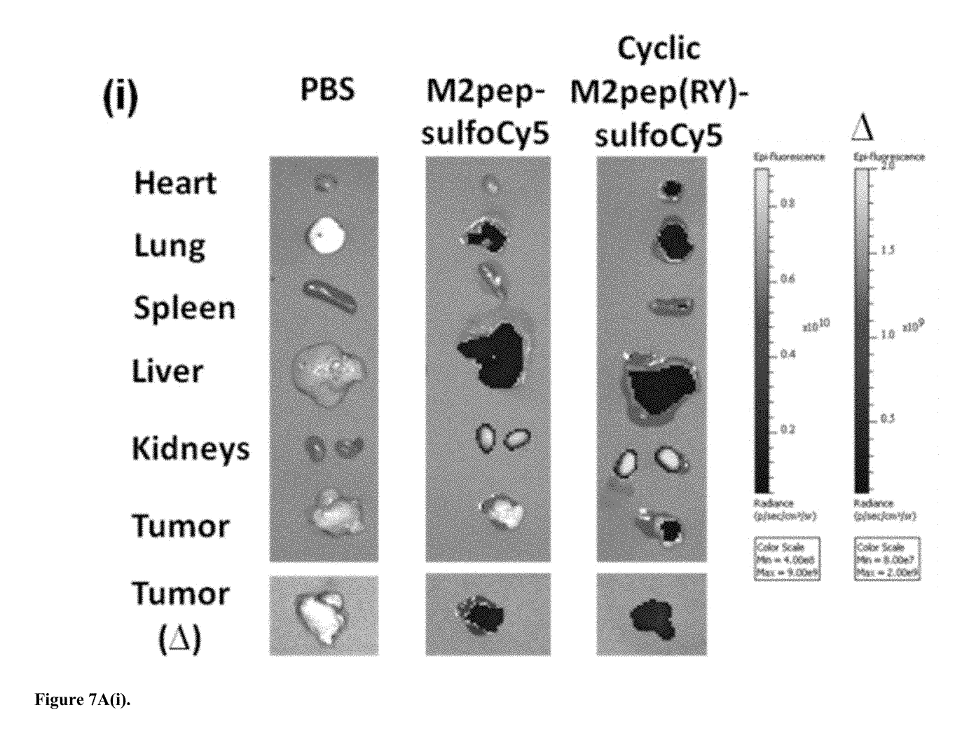

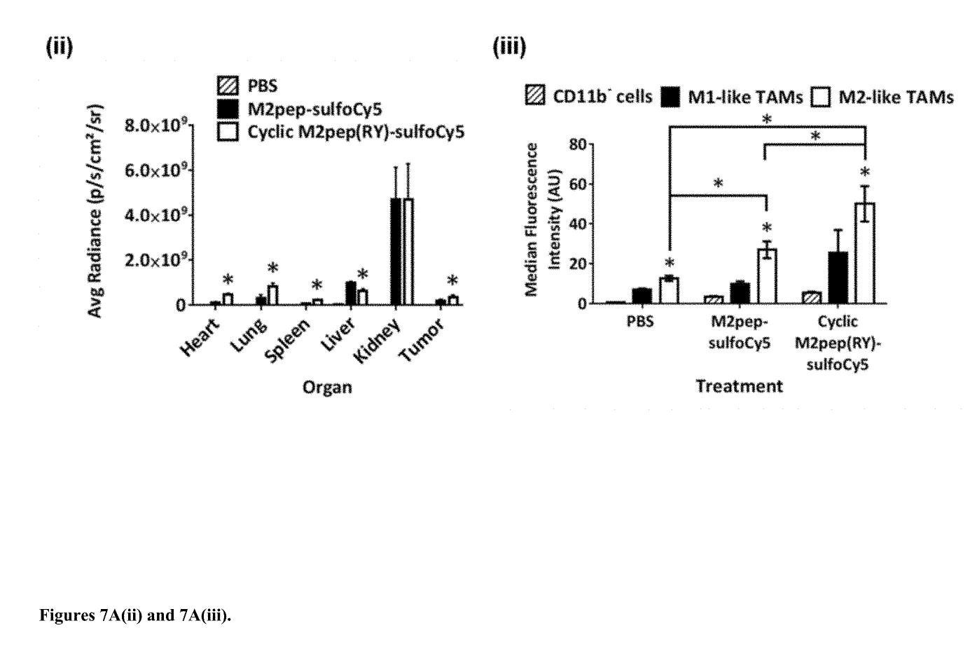

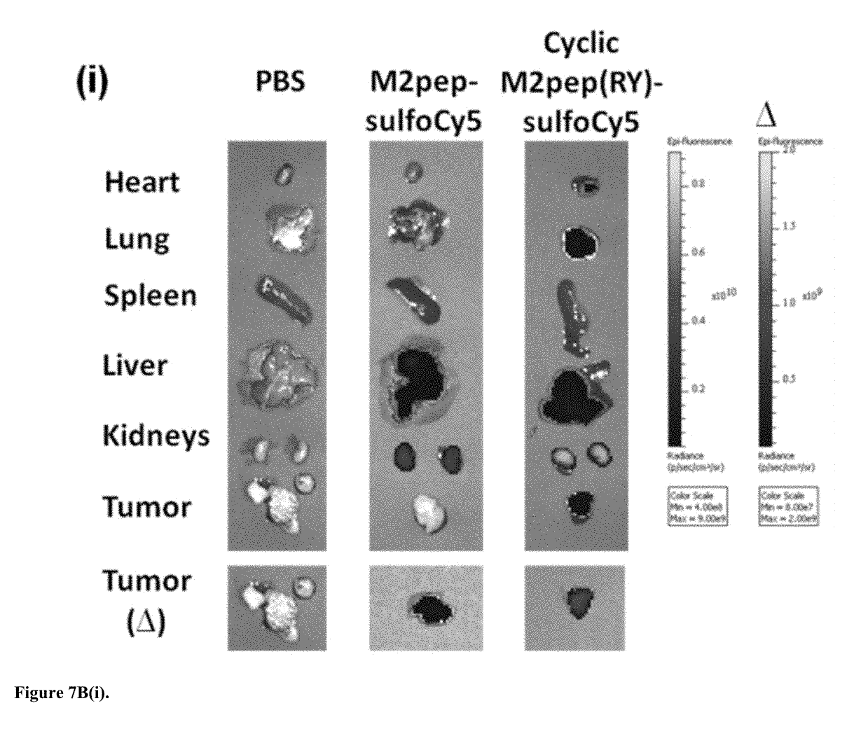

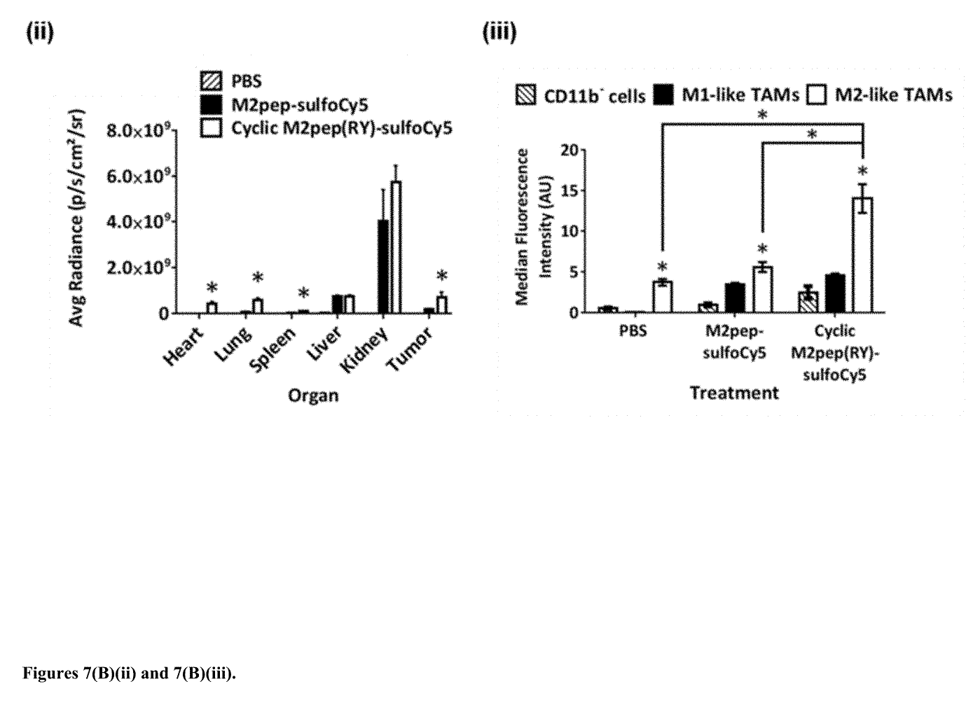

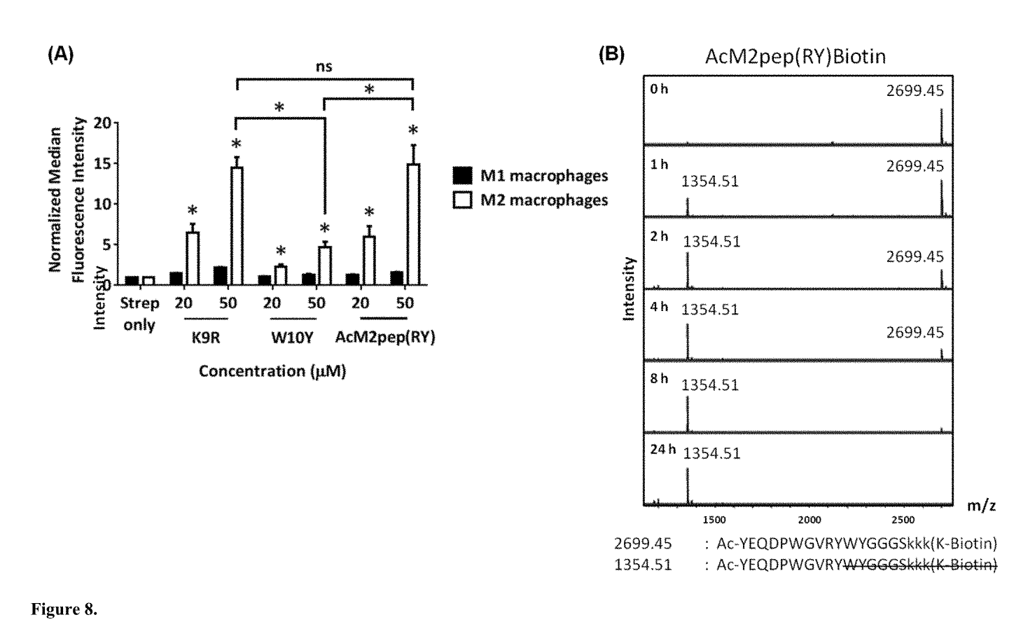

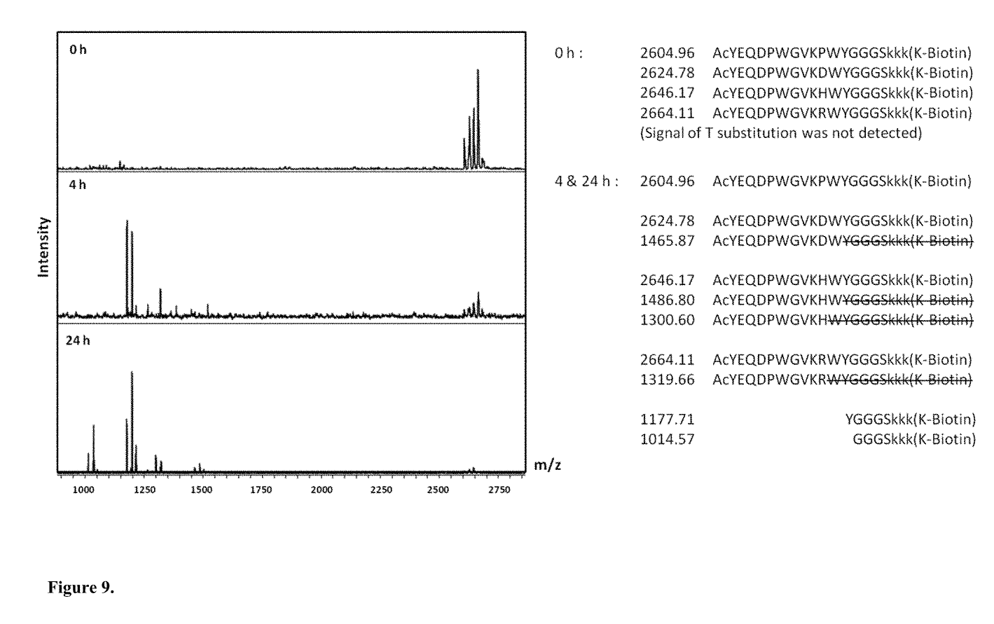

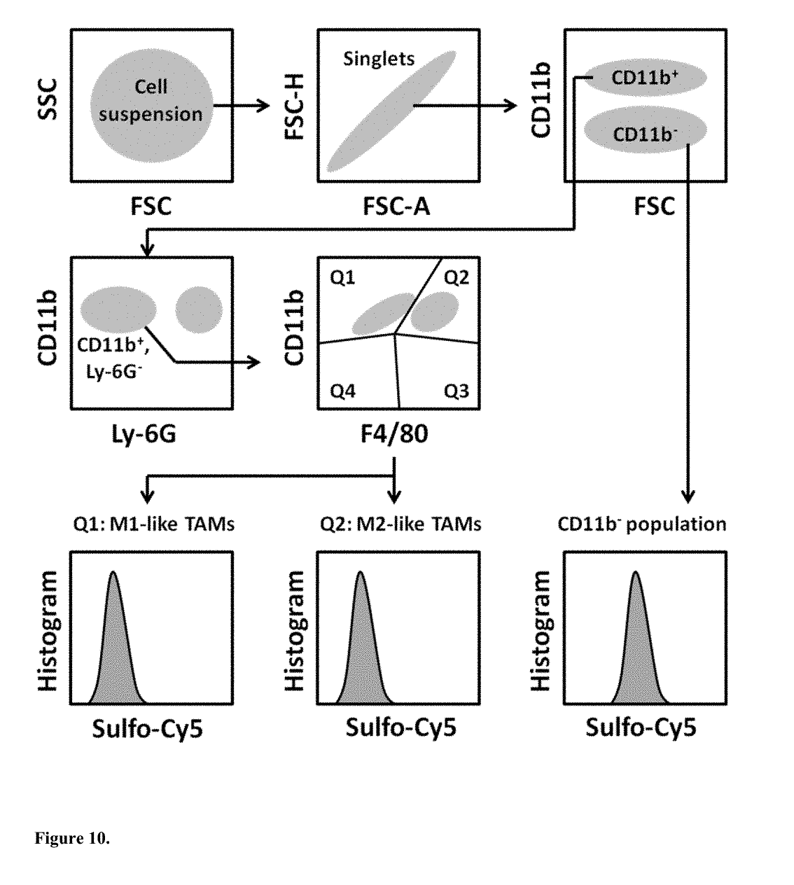

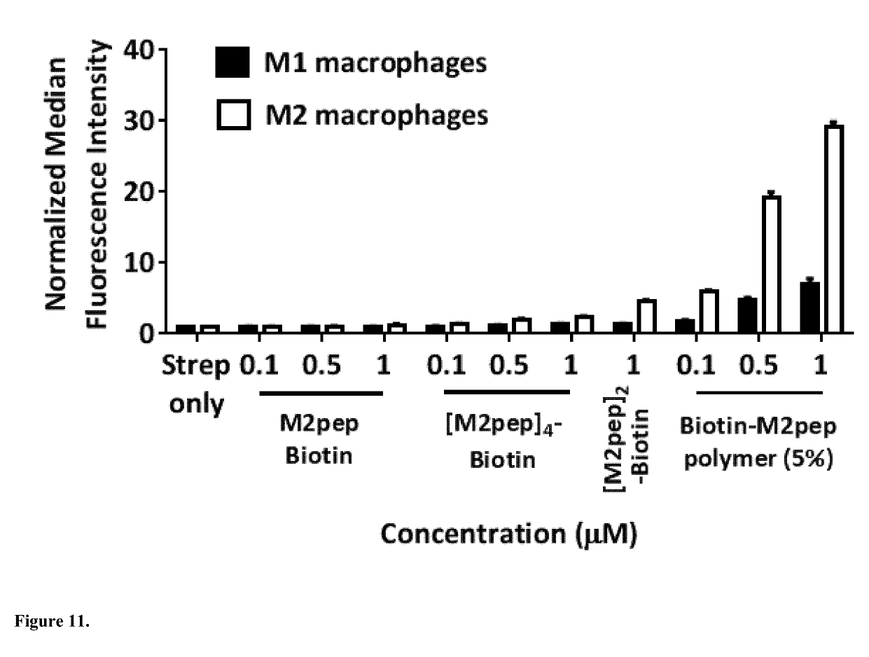

We assessed the effectiveness of cyclic M2pep(RY) as an in vivo TAM-targeting ligand in a syngeneic CT-26 colon carcinoma and an orthotopic 4T1 breast carcinoma tumor model. The biodistribution study was first performed in the CT-26 tumor model which we previously used to evaluate the original M2pep [14]. CT-26 tumor-bearing mice were intravenously administered with PBS control, M2pep-sulfoCy5, or cyclic M2pep(RY)-sulfoCy5 and perfused 20 min after. Organs were then harvested and imaged under the Xenogen IVIS. Subsequently, the tumors were processed into cell suspension, stained with antibodies, and analyzed for intratumoral distribution by flow cytometry. The gating strategy is depicted in Fig S3. In general, higher accumulation of cyclicM2pep(RY)-sulfoCy5 than M2pep-sulfoCy5 in tumors and other organs was observed with an exception of liver and kidneys (FIG. 7A(i-ii)). The highest fluorescent signal was observed in kidneys where the peptides were filtered and excreted. Intratumorally, cyclic M2pep(RY)-sulfoCy5 promoted significantly higher uptake in M2-like TAMs compared to M2pep-sulfoCy5 while selectivity to M2-like TAMs over M1-like TAMs and non-TAM CD11b⁻ cells was observed in both peptide treatments (FIG. 7A(iii)).

Following successful application of cyclic M2pep(RY) for targeting M2-like TAMs in CT-26 model, we further evaluated its effectiveness as an in vivo targeting ligand in 4T1 breast tumor model since high accumulation of M2-like TAMs in breast cancer has been correlated to poor disease prognosis [32]. As observed in the CT-26 tumor model, cyclic M2pep(RY)-sulfoCy5 localized to 4T1 tumors to a greater extent compared to M2pep-sulfoCy5 (FIG. 7B(i-ii)), and also accumulated preferentially in M2-like TAMs (FIG. 7B(iii)). Hence, we have demonstrated through the studies of two tumor models that cyclic M2pep(RY) can be effectively used as an in vivo M2-TAM-targeting ligand due to its superior serum stability and enhanced binding affinity over the original M2pep. Its utility is expected to extend across multiple tumor models. In perspective, cyclic M2pep(RY)-mediated delivery of therapeutic cargos or imaging agents to M2-like TAMs could enable scientists to develop a more potent and selective M2-like TAM immunomodulation regimen or to better monitor their behavior over the course of tumor development.

### Synthesis and Characterization of M2 Macrophage Binding-Polymers

All materials for M2 Macrophage-binding polymer (M2P) backbone synthesis were purchased from Sigma-Aldrich. The M2P backbone with a target composition of 80% (hydroxyethyl) methacrylate (HEMA) and 20% N-hydroxysuccinimide methacrylate (NHSMA) was synthesized using RAFT polymerization. For a typical synthesis, 310 uL HEMA (2.56 mmol), 117.2 mg NHSMA (0.640 mmol), 1 mL AIBN (0.876 mg/ml in DMAc, 0.0053 mmol), and 4.47 mg CPADB (0.016 mmol) were dissolved in a 5-mL reaction vessel with 3.02 mL DMAc for a final monomer concentration of 0.6 M. The reaction mixture was purged with argon for 10 minutes and reacted under stirring conditions at 70° C. for 24 hr. Fluorescent M2 Macrophage-binding polymer (fM2P) was synthesized by altering the composition of M2P to 78% HEMA and 2% fluorescein O-methacrylate and maintaining 20% NHSMA. Polymers were precipitated in ether, dissolved in DMAc, and re-precipitated in ether to remove unreacted monomers. Polymers were then dried and stored in a vacuum-sealed oven. Dithiobenzoate groups were removed in a subsequent reaction with a 20:1 molar ratio of AIBN to polymer. Transition of the solution from pink to clear was a positive indicator that the end-capping reaction was near or at completion. Degree of polymerization and monomer composition were determined using H1 NMR, and polydispersity and molecular weight were measured using GPC. The absence of CTA peaks on H1 NMR was used to confirm removal of dithiobenzoate groups. M2 Macrophage-binding peptides were conjugated to NHS reactive groups on M2P via the ε-amine on the C-terminus lysine under organic basic reaction conditions as reported by Yanjarappa et al [Yanjarappa 2006]. After 24 hr, reaction solutions were transferred to snakeskin dialysis tubing with 10 kDa MWCO and dialyzed against PBS for 2 days to remove free peptide. Polymers were then dialyzed against DI water for 2 days to remove salts from the PBS and lyophilized. The number of peptides per polymer was determined by measuring absorption at 280 nm using a Nanodrop 2000 UV-vis spectrophotomer.

### Synthesis of Biotin-p(HPMA-co-AziMA)

In this example, Biotin-PEG3-CTP was used as a chain transfer agent (CTA) for RAFT polymerization. Biotin-PEG3-CTP was synthesized by reacting 4-Cyano-4-(phenylcarbonothioylthio)pentanoic acid N-succinimidyl ester (NHS-CTP, 2 eq.) with Amine-PEG3-Biotin (EZ-Link™, ThermoFisher Scientific, 1 eq. added in 3 portions) in DIPEA (4 eq.) and DCM for 48 h before purification by flash column chromatography and RP-HPLC. CTA (dissolved in ethanol), VA-044 initiator, HPMA, and APMA were dissolved in 9:1 (v/v) 1 M acetate buffer (pH 5.1): ethanol at the [CTA]:[I]:[HPMA]:[APMA] ratio of 1:0.5:390:10 and the final monomer concentration of 1 M. The relative ratio of [CTA]:[HPMA]:[APMA] may be varied to obtain polymers with different degree of polymerization and functionalizable monomer content for peptide grafting. The reaction solution in a flask was sealed with a rubber septum and purged with nitrogen for 15 min before being immerged in an oil bath preheated to 44° C. The polymerization was stopped after 24 h by air exposure and then precipitated in cold acetone twice. Endcapping of polymer was performed by reacting the polymer with excess of VA-044 (40 eq.) in 1 M acetate buffer (pH 5.1) at 44° C. for 4 h with subsequent purification by dialysis against water and lyophilization. Conversion of amine groups on APMA to azide groups was performed via diazo-transfer reaction using imidazole-1-sulfonyl azide hydrochloride (3 APMA eq.) in DIPEA (9 APMA eq.) and DMSO with CuSO₄.5H₂O (0.01 APMA eq.) as a catalyst. The reaction was stirred at room temperature for 24 h before dialysis against water and lyophilization to obtain Biotin-p(HPMA-co-AziMA). Alternatively, azidopropyl methacrylamide monomer may be synthesized and directly copolymerized with HPMA to obtain the same product.

### Synthesis of DFBP-cyclic M2pep(RY)Alkyne

The peptide precursor on resin (CGYEQDPWGVRYWYGCkkk(K-Alkyne); (SEQ ID NO:56)) was synthesized following Fmoc/tbu solid-phase peptide synthesis with standard protecting groups except the cysteines where Stbu and Mmt protecting groups were used for the N-terminal and C-terminal cysteines respectively. The Stbu protection group was deprotected by an overnight incubation in a solution of DTT (10 eq.) and TEA (20 eq.) in DMF. The free cysteine was then functionalized by incubating in a solution of DFBP (4 eq.) and TEA (8 eq.) in DMF for 3 h. Mmt protecting group was next removed by 20-min incubations in TFA/TIPS/DCM (2:5:93 v/v/v) until the yellow-colored solution disappeared (3-4 incubations). The DFBP cyclization was subsequently conducted by incubating the peptide resin in a TEA solution (8 eq.) in DMF for 24 h. The cyclized peptide was cleaved and purified by RP-HPLC. All deprotection and DFBP functionalization steps were monitored by the modified on-resin Ellman's assay following the previously reported protocol.
Grafting of DFBP-Cyclic M2pep(RY)Alkyne onto Biotin-p(HPMA-Co-AziMA)
Biotin-p(HPMA-co-AziMA) (1 eq.) and DFBP-cyclic M2pep(RY)Alkyne (2 eq.) were dissolved in 1:1 (v/v) DMF:H₂O. A pre-complexed solution of tris(3-hydroxypropyltriazolylmethyl)amine (THPTA, 5 eq.) and CuSO₄.5H₂O (5 eq.) was added to the solution followed by sodium ascorbate (5 eq.). The reaction solution was purged with nitrogen for 15 min and stirred for 3 h at 37° C. The product was dialyzed against water for 3 days and lyophilized.

### Engineering an Affinity-Enhanced Peptide Through Optimization of Cyclization Chemistry

Cyclization optimization of an M2 “anti-inflammatory” macrophage-binding peptide (M2pep) resulted in a significant increase in binding affinity of the optimized analog to M2 macrophages while maintaining binding selectivity compared to M1 “pro-inflammatory” macrophages. Four cyclic M2pep(RY) analogs with diverse cyclization strategies were synthesized and evaluated; 1) Asp-[Amide]-Lys, 2) Azido-Lys-[Triazole (Copper(I)-catalyzed alkyne-azide cycloaddition (CuAAC))]-Propargyl-Gly, 3) Cys-[Decafluorobiphenyl (DFBP)]-Cys, and 4) Cys-[Decafluorobiphenyl sulfone (DFS)]-Cys, whereby the chemical entity/linker at the linkage site is shown in the square bracket and is between the residues involved in cyclization. These peptides were compared to a disulfide-cyclized M2pep(RY), a serum-stable, affinity enhanced analog to linear M2pep. DFBP-cyclized M2pep(RY) exhibited the highest binding activity to M2 macrophages with apparent dissociation constant (KD) about 2.03 μM compared to 36.3 μM for the original disulfide-cyclized M2pep(RY) and 220 μM for the original linear peptide. DFS-cyclized M2pep(RY) also binds more strongly than the original cyclized analog whereas Amide- and Triazole-cyclized M2pep(RY) analogs bind less strongly. DFBP alone has negligible binding to M2 macrophages and the incorporation of diphenylalanine to the original sequence improves binding activity at the expense of solubility and increased toxicity.

### Peptide Synthesis

In this work, five cyclic M2pep(RY) peptides were synthesized using various cyclization chemistries. Successful synthesis of all peptides was verified by MALDI-ToF MS. On-resin cyclization of Amide-, Triazole-, and DFBP-cyclized M2pep(RY)Biotin proceeded efficiently based on HPLC traces during purfication. Successful CuAAC-mediated cyclization of Triazole-cyclized M2pep(RY)Biotin was confirmed by insensitivity to TCEP-mediated azide reduction and inactivity to subsequent CuAAC reaction with propargyl alcohol. Attempts in on-resin cyclization of DFS-cyclized M2pep(RY)Biotin following the developed strategy for on-resin synthesis of DFBP-cyclized M2pep(RY)Biotin were not successful and instead, a major side product of −346 Da was observed. Nonetheless, in-solution cyclization of DFS-cyclized M2pep(RY)Biotin was optimized to proceed in high yield with minimal formation of an unidentified “−20 Da” side product. In addition, we found that addition of TCEP to the DFBP and DFS cyclization reactions in solution significantly increases yield of the desired product by suppressing formation of the disulfide-cyclized peptide. Three of the cyclized variants (Disulfide, DFBP, and DFS analogs) can be readily synthesized from the same linear precursor, thus saving time for the optimization process. Although not investigated in this study, it should also be possible to synthesize Amide- and Triazole-cyclized analogs from a common linear precursor, followed by coupling with Boc-Propargylglycine-OH for CuAAC-mediated cyclization and coupling with Boc-Asp(OAll)-OH followed by reduction of azide to amine and OAll deprotection before on-resin lactamization, respectively.

### Materials

Protected amino acids, 2-(6-chloro-1H-benzotriazole-1-yl)-1,1,3,3-tetramethylaminium hexafluorophosphate (HCTU), (benzotriazol-1-yloxy)tripyrrolidinophosphonium hexafluorophosphate (PyBOP), and 1-hydroxybenzotriazole hydrate (HOBt) were purchased from AAPPTec (Louisville, Ky.), AnaSpec (Fremont, Calif.), and Chem-Implex International (Wood Dale, Ill.). Triisopropylsilane (TIPS), 1,2-ethanedithiol (EDT), 1,3-dimethoxybenzene (DMB), tetrakis(triphenylphosphine)palladium(0) (Pd(PPh3)4), sodium diethyldithiocarbamate trihydrate, copper(II) sulfate pentahydrate (CuSO4.5H2O), and tris(2-carboxyethyl)phosphine hydrochloride (TCEP) were purchased from Sigma-Aldrich (St. Louis, Mo.). Tris[(1-benzyl-1H-1,2,3-triazol-4-yl)methyl]amine (TBTA) was purchased from TCI America (Portland, Oreg.). Normal mouse serum (Catalog number 10410), phenylsilane, decafluorobiphenyl, and other reagents were purchased from Thermo Fisher Scientific (Waltham, Mass.). Quant*Tag Biotin quantification kit was purchased from Vector Laboratories (Burlingame, Calif.). Imidazole-1-sulfonyl azide hydrochloride and decafluorobiphenyl sulfone (DFS) were synthesized following previously reported protocols. For the synthesis of DFS, an additional purification step was performed by dissolving the partially purified light yellow product in DMF and precipitating in H₂O twice before lyophilizing to obtain the product as white powder.

### Synthesis of Biotinylated M2pep Analogs

General Peptide Synthesis Strategy

Peptides were synthesized using a PS3 automated peptide synthesizer (Protein Technologies, Phoenix, Ariz.) via standard Fmoc/tbu solid-phase peptide synthesis unless noted below:

Linear precursor:
X₃GYEQDPWGVRYWYGX₃kkk(K-Biotin)

| M2pep analogs | Protected X₁ | Protected X₂ |
|---|---|---|
| Disulfide-cyclized | Fmoc-Cys(tbu)—OH | Fmoc-Cys(tbu)—OH |
| M2pep(RY)Biotin | | |
| Amide-cyclized | Fmoc-Asp(OAll)-OH | Fmoc-Lys(Alloc)-OH |
| M2pep(RY)Biotin | | |
| Triazole-cyclized | Boc-Lys(Fmoc)—OH | Fmoc-Propargyl-Gly-OH |
| M2pep(RY)Biotin | | |
| DFBP-cyclized | Fmoc-Cys(Stbu)—OH | Fmoc-Cys(Mmt)-OH |
| M2pep(RY)Biotin | | |
| DFS-cyclized | Fmoc-Cys(tbu)—OH | Fmoc-Cys(tbu)—OH |
| M2pep(RY)Biotin | | |

Manual coupling of amino acids was performed by incubating the peptide resin in a solution of amino acid (4 eq.) and HCTU (3.9 eq.) in 0.4 M Nmethylmorpholine in DMF for 3 h. Fmoc deprotection was performed by two 30-min incubations in 20% (v/v) piperidine in DMF. Biotinylated resin was prepared from NovaPEG rink amide resin as reported previously and was used for the synthesis of all biotinylated peptides. Peptides were cleaved off the resin by incubating in a cleavage cocktail containing TFA/DMB/TIPS/EDT (90:5:2.5:2.5 v/v/v/v) for 2.5 h followed by double precipitation in cold ether. EDT was included in the cleavage cocktail only for peptides containing a free cysteine after cleavage. The crude peptides were purified by RP-HPLC (Agilent 1200, Santa Clara, Calif.) to 95% purity using a Phenomenex Fusion-RP C18 semi-preparative column (Torrance, Calif.) at the flow rate of 5 mL/min with H₂O (0.1% TFA) and ACN (0.1% TFA) as a mobile phase. Molecular weights of the purified peptides were confirmed by MALDI-ToF Mass Spectrometry (MS).

Disulfide-Cyclized M2pep(RY)Biotin

The purified linear precursor (10-20 mg) was dissolved in H₂O (1-3 mL) and added to a deaerated 0.1 M ammonium bicarbonate buffer (pH 8) at a final peptide concentration of 0.14 mg/mL. The solution was left to stir for 2 days for oxidation, then acidified with TFA and desalted with a HyperSep™ C18 cartridge. The desalted cyclized peptide was concentrated with rotary evaporator and lyophilized.

Amide-Cyclized M2pep(RY)Biotin

The N-terminus-Fmoc-protected precursor peptide on resin was incubated in a solution of Pd(PPh3)4 (0.2 eq.) and phenylsilane (20 eq.) in DCM for 20 min twice to selectively deprotect OAll and Alloc protecting groups. The resin in the deprotection solution was purged with nitrogen for 5 min prior to each incubation. Following deprotection, the resin was washed with 5 mM sodium diethyldithiocarbamate solution in DMF to remove the palladium catalyst. Onresin lactamization follows the optimized protocol by Thakkar et al. by incubating the resin in a solution of HOBt (5 eq.), PyBOP (5 eq.), and DIPEA (10 eq.) in DCM/DMF/MNP (3:2:2 v/v/v) with 1% (v/v) triton X100 for 24 h. A small amount of peptide on resin was cleaved to monitor for completion of the cyclization via MALDI-ToF MS. Upon completion, the cyclized peptide was Fmoc-deprotected, cleaved, and purified by RP-HPLC.

Triazole-Cyclized M2pep(RY)Biotin

The epsilon amine on the N-terminal lysine of the precursor peptide on resin was Fmoc-deprotected and converted to azide by an overnight incubation in a solution of imidazole-1-sulfonyl azide hydrochloride (3 eq.), CuSO4.5H2O (0.01 eq. pre-dissolved in a small amount of H2O), and DIPEA (9 eq.) in DMSO as reported previously. The completion of the diazotransfer reaction was monitored by the Kaiser test for the absence of amine. Alternatively, Fmoc-Lys(N3)-OH may be used instead of Boc-Lys(Fmoc)-OH. On-resin CuAAC cyclization was performed by incubating the peptide resin in a solution of tetrakis(acetonitrile)copper(I) hexafluorophosphate (1 eq.), TBTA (1 eq.), and DIPEA (2 eq.) in DMF for 24 h. The resin was then washed with 5 mM sodium diethyldithiocarbamate solution in DMF, and the cyclized peptide was cleaved and purified by RP-HPLC.

DFBP-Cyclized M2pep(RY)Biotin

In-solution cyclization of the linear peptide precursor with DFBP follows the previously described protocol modified with an addition of TCEP to suppress disulfide formation during DFBP cyclization. In brief, the crude peptide precursor (20 mg) was dissolved in DMF (2.5 mL) in a 5-mL microcentrifuge tube. A fresh TCEP stock solution in H₂O (5 eq.) was added to the peptide solution, and an additional volume of H₂O was added to make up a total H₂O volume of 0.5 mL. The reaction was left for 10 min followed by an addition of 50 mM tris base in DMF (1.5 mL). DFBP predissolved in DMF (2 eq.) was then added, and the reaction was vortexed vigorously. On the next day, the reaction was diluted with 3-fold volume of H₂O (0.1% TFA) and desalted with a Sep-Pak C18 cartridge. The desalted cyclized peptide solution was concentrated and purified by RP-HPLC. On-resin cyclization of DFBP utilizes two cysteines with orthogonal protecting groups. First, the Stbu protecting group was deprotected by an overnight incubation in a solution of DTT (10 eq.) and TEA (20 eq.) in DMF. The free cysteine was functionalized by incubating in a solution of DFBP (4 eq.) and TEA (8 eq.) in DMF for 3 h. Mmt protecting group was then removed by 20-min incubations in TFA/TIPS/DCM (2:5:93 v/v/v) until yellowcolored solution disappeared (3-4 incubations). The DFBP cyclization was then proceeded by incubating the peptide resin in a TEA solution (8 eq.) in DMF for 24 h. The cyclized peptide was cleaved and purified by RP-HPLC. All deprotection and DFBP functionalization steps were monitored by the modified on-resin Ellman's assay following previously reported protocols.

DFS-Cyclized M2pep(RY)Biotin

In-solution cyclization of the linear peptide precursor with DFS was performed as described for DFBP cyclization with an exception that the reaction was quenched by an addition of 3-fold volume of H₂O (0.1% TFA) after a 10-min incubation to prevent formation of a side product.

### Bone Marrow Harvest and Macrophage Culture

All animal handling protocols were approved by the University of Washington Institutional Animal Care and Use Committee. Bone marrow-derived cells were harvested from female c57bl/6 mice as previously described. In brief, femurs and tibias of the mice were excised and flushed with RPMI 1640 medium via an 18G needle. The cells were cultured on petri dishes containing RPMI 1640 medium supplemented with 20% donor horse serum, 1% antibioticantimycotic (AbAm), and 20 ng/mL M-CSF. After 7 d in culture, the medium was replaced with an activation medium whereby M-CSF was replaced with 25 ng/mL IFN-γ and 100 ng/mL LPS for M1 activation or with 25 ng/mL IL-4 for M2 activation. For M1 activation medium, AbAm was replaced with 1% penicillin-streptomycin (Pen-Strep). After 2 d of activation, the activated macrophages were scraped off the petri dishes for binding studies.

### In Vitro Binding Study

Biotinylated peptide stock solutions were prepared in H₂O at 10 mg/mL concentration and quantified for the exact concentration using Quant*Tag Biotin quantification kit. The stock solutions were diluted in PBS with 1% BSA (PBSA) to different concentrations for binding studies. Activated macrophages were seeded on a black 96-well plate (50,000 cells/well) and incubated with the peptide solutions for 20 min on ice. The cells were washed twice with PBSA and subsequently incubated with streptavidin-FITC for 15 min on ice. After two additional washes, the cells were resuspended in PBS for analysis via MACSQuant Flow Cytometer (Miltenyi Biotec, San Diego, Calif.). Propidium iodide (PI) was added before running each sample to assess for cell viability. Data analysis was processed on FlowJo Analysis Software (Tree Star, Ashland, Oreg.). Construction of binding curves and determination of apparent KD were performed using GraphPad Prism 6 (GraphPad Software Inc., La Jolla, Calif.) based on an average normalized median fluorescence intensity value of triplicate samples in the same experiment.

### CD Measurement

M2pep analogs were prepared in H₂O at 50 μM concentration. CD measurement of the peptides was performed on Jasco 720 Circular Dichroism Spectrophotometer (Jasco Inc., Easton, Md.) at 25° C. with an average from 8 scans. The CD spectra were corrected for baseline and smoothened on Spectra Manager Software (Jasco Inc., Easton, Md.).

### In Vitro Serum Stability Study

Different M2pep analogs (30 μL from 10 mg/mL stock solution in H₂O) were incubated in normal mouse serum (300 μL) at 37° C. in an incubator. An aliquot of the serum (40 μL) was drawn at different time intervals and precipitated with an equal volume of cold ACN. The mixture was centrifuged at 15,000 rpm for 5 min, and the supernatant was collected. A solution of H2O/ACN (1:1 v/v, 80 μL) was added to the pellet with a 10-min sonication to further extract residual peptides. The mixture was then centrifuged. The supernatant was pooled with the former and dried under vacuum on a Speedvac machine. The peptide pellet was resuspended in H2O (50 μL) with a 10-min sonication before analysis by MALDI-ToF MS.

### In Vitro Binding Study

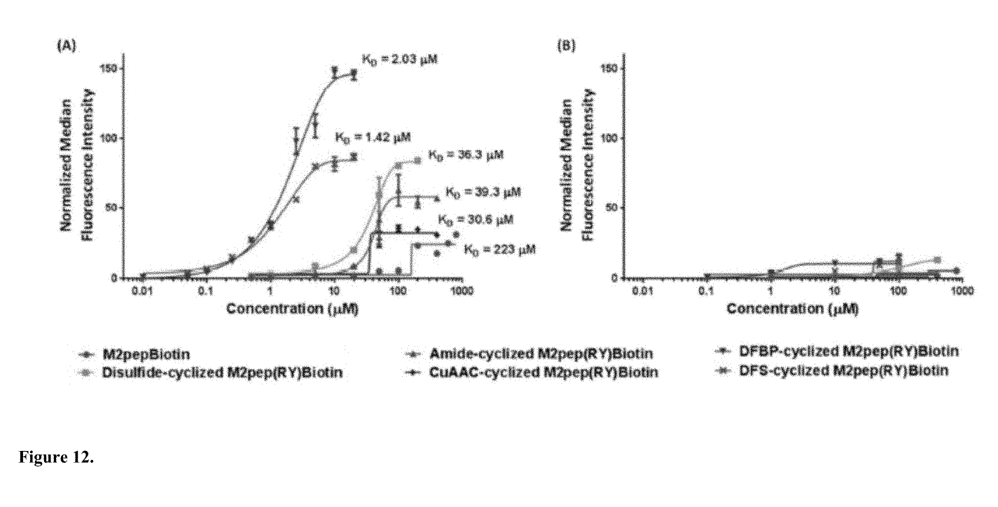

M1 and M2 polarized murine macrophages were incubated with various concentrations of biotinylated M2pep analogs and probed for peptide binding using streptavidin-FITC with flow cytometry analysis. Binding curves were acquired by determining the median fluorescence intensity (MFI) of streptavidin-FITC associated with cells at various peptide concentrations, and apparent KD values were calculated based on half-maximal MFI of each individual peptide analog. All M2pep analogs bind preferentially to M2 macrophages over M1 macrophages (FIG. 12). For comparison, binding curves for M2pepBiotin and Disulfide-cyclized M2pep(RY)biotin are also included. Disulfide-, Amide-, and Triazole-cyclized M2pep(RY)Biotin have similar KD values, between 30-40 μM, although varying maximum MFI values were observed for each construct. Strikingly, DFBP- and DFScyclized M2pep(RY)Biotin have significantly improved affinity with about 15-30 fold lower KD values (2.03 μM and 1.42 μM, respectively) than the other cyclic analogs. Comparing these two analogs, DFS-cyclized M2pep(RY)Biotin has lower maximal MFI than the DFBP analog. The maximum MFI correlates to the maximal extent at which peptides can engage with surface receptors on plasma membrane of target cells. It is surprising to us that different cyclic analogs exhibit different maximal MFI values even though they are expected to bind to the same target receptor on M2 macrophages. However, since the identity of the M2pep receptor remains unknown, we are not able to make further conclusions regarding the mechanism of binding of these peptide constructs. Nonetheless, our binding data demonstrate that optimization of cyclization chemistry is important in maximizing the cyclic peptide activity

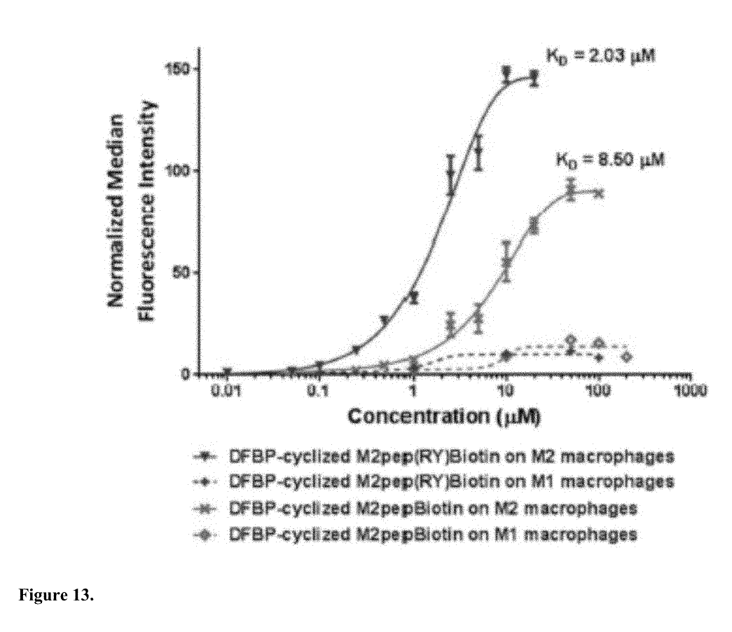

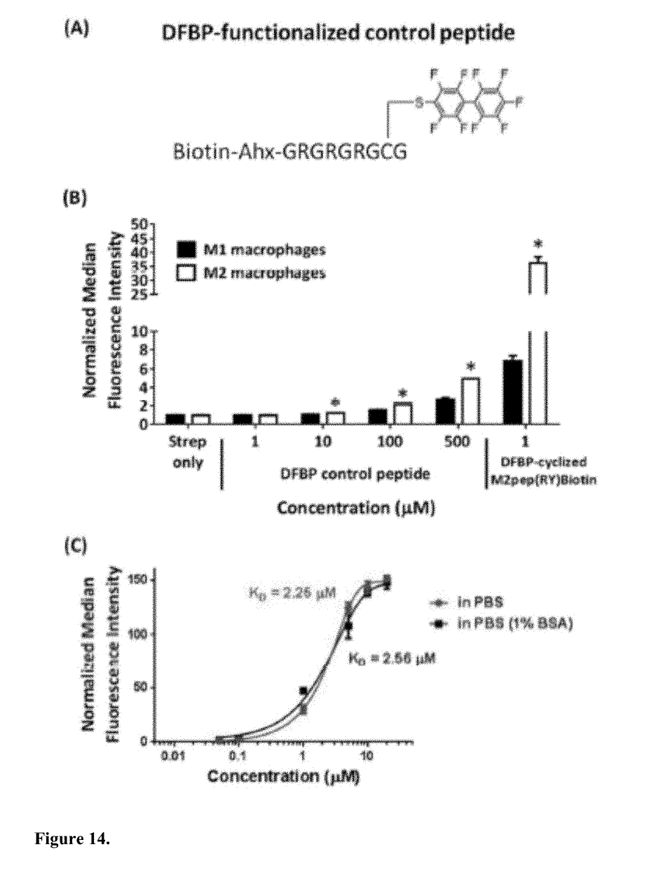

Due to the low KD and high maximal MFI obtained for DFBP-cyclized M2pep(RY)Biotin, we next verified whether the RY modification, originally reported to improve binding affinity of M2pep, remains a relevant modification for this cyclic analog. DFBP-cyclized M2pepBiotin without RY modification was synthesized and evaluated for binding activity. Binding studies confirmed that the RY modification enhances binding activity of the cyclized-M2pep peptide to M2 murine macrophages by about 4-fold compared to the original sequence (FIG. 13). Hence, we confirmed the discovery of DFBP-cyclized M2pep(RY)Biotin as an affinity-enhanced M2 macrophage-targeting peptide which could be useful for future development of diagnostics or drug delivery systems targeting M2 macrophages or M2-like tumor-associated macrophages. In order to understand the affinity enhancement of DFBP-cyclized M2pep(RY)Biotin, we first assessed the effect of DFBP on macrophage binding by synthesizing a DFBP-functionalized linear control peptide (Biotin-Ahx-GRGRGRG(C-DFBP)G) where DFBP is conjugated to a single cysteine and the GR region serves as a spacer and solubility enhancer for DFBP (FIG. 14A). This peptide binds very weakly to macrophages even at 100 μM whereas DFBP-cyclized M2pep(RY)Biotin exhibits strong binding activity even at 1 μM (FIG. 14B). This data suggests that DFBP itself does not increase binding to the unknown M2pep receptor and that DFBP may instead increase binding by an alternative mechanism such as via altering or stabilizing peptide conformation. Since DFBP is a small hydrophobic molecule, we speculated that DFBP might bind to some hydrophobic pockets on BSA present in our binding solutions and promote enhanced binding via a multivalent effect. This possibility was eliminated based on binding studies of DFBP-cyclized M2pep(RY)Biotin performed in both PBS and PBSA (PBS with 1% albumin) that showed no difference in binding (FIG. 14C).

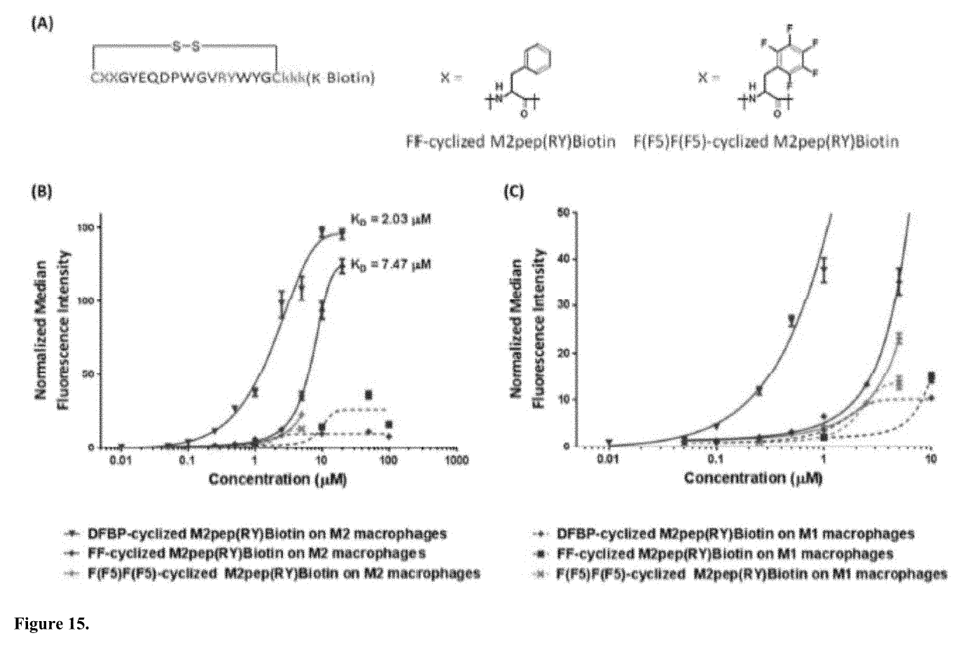

Next, we investigated whether the higher binding activity of DFBP-cyclized M2pep(RY)Biotin could be recapitulated by an addition of a dipeptide with aromatic side chain into the original disulfide-cyclized M2pep(RY)Biotin peptide. Two additional disulfide-cyclized M2pep(RY)Biotion analogs were synthesized incorporating either diphenylalanine (FF-cyclized M2pep(RY)Biotin) or dipentafluorophenylalanine (F(F5)F(F5)-cyclized M2pep(RY)Biotin) before the N-terminal cysteine to assess the effect of aromatic ring and fluorine substitution on binding activity to macrophages (FIG. 15A). Introduction of these dipeptides significantly reduces solubility of the peptide analogs with the F(F5)F(F5)-cyclized analog being less soluble than the FF-cyclized analog. Notably, despite being highly fluorinated, DFBP-cyclized M2pep(RY)Biotin has higher solubility than FF-cyclized M2pep(RY)Biotin. Addition of diphenylalanine indeed improves binding activity of the peptide on M2 macrophages implying that the presence of aromatic structure near the site of cyclization may contribute to the enhanced binding (FIG. 15B). F(F5)F(F5)-cyclized M2pep(RY)Biotin was very toxic to macrophages even at low micromolar concentrations, so we were not able to accurately construct a binding curve for this analog. Nonetheless, based on the binding data at the lower concentrations (FIG. 15C), this analog seems to exhibit similar binding activity as FF-cyclized M2pep(RY)Biotin with M2 macrophages implying that fluorine substitution may not have a significant effect towards binding activity. However, since FF-cyclized M2pep(RY)Biotin still has lower binding activity than DFBP-cyclized M2pep(RY)Biotin, there are likely additional factors involved in the enhanced binding phenomenon observed with the DFBP-cyclized peptide.

### Circular Dichroism (CD) Measurement

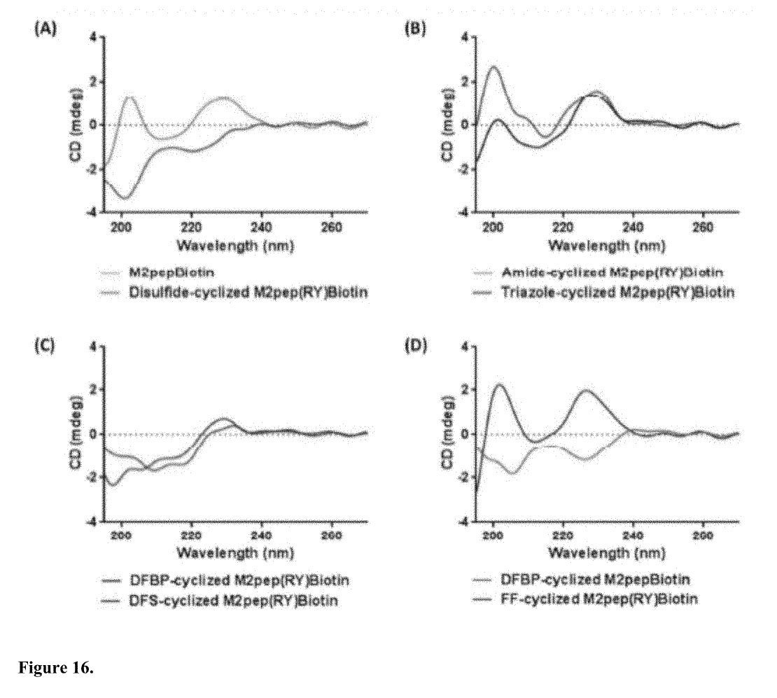

Improvement in peptide activity via cyclization can sometimes be attributed to reinforcement in the peptide's secondary structure. Hence, we performed a preliminary assessment on peptide conformation of the M2pep peptides via CD measurement. None of the CD spectra of these M2pep analogs matches characteristic spectra of 100% α-helix, β-sheet, or random coil structures 32 implying that the peptides may only be partially structured or unstructured (FIG. 16A-D). The effect of RY modification results in an inversion from a predominantly negative CD signal with two minima at about 200 and 225 nm to a predominantly positive signal with two maxima at about 200 and 230 nm (FIG. 16A). A decline in the CD signal at 200 and 230 nm maxima for DFBP- and DFS-cyclized M2pep(RY)Biotin analogs was observed (FIG. 16C) compared to the other cyclic M2pep(RY)Biotin analogs which may account for the affinity improvement of the former. However, this trend is not observed in FF-cyclized M2pep(RY)Biotin which also exhibits enhanced affinity. Unlike stapled peptides of helical nature whose secondary structure reinforcement may be estimated in term of helicity content, it is more challenging to elucidate the conformational reinforcement of non-helical peptides like cyclic M2pep(RY) analogs. Future characterization studies on identification of M2pep receptor as well as its interaction with M2pep peptides will be needed in order to better understand the observed enhanced affinity to M2 macrophage.

### In Vitro Serum Stability Study

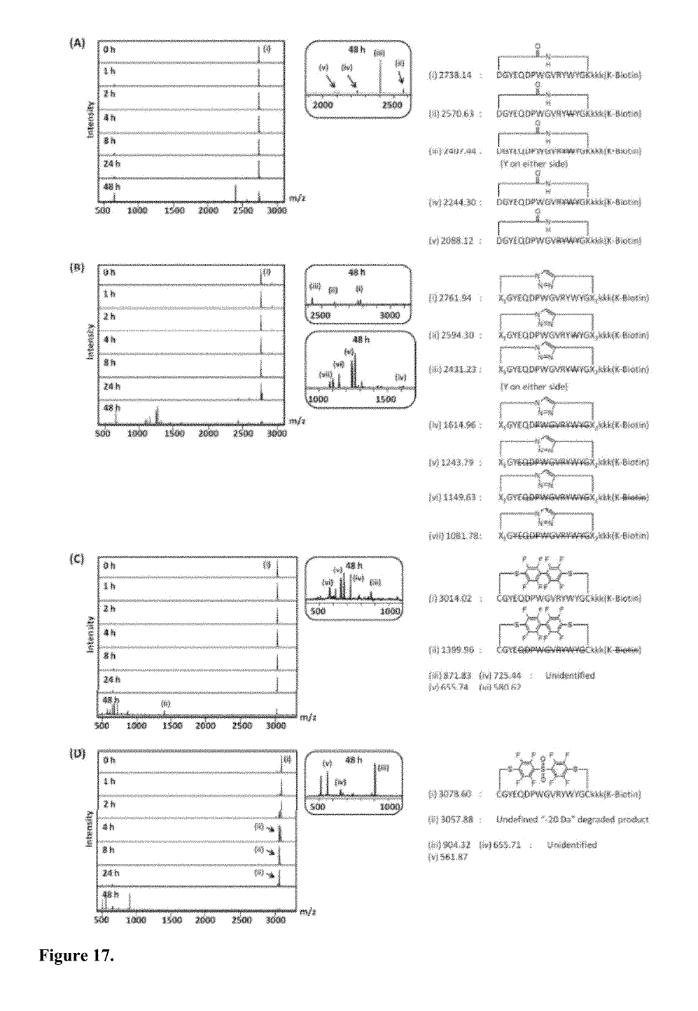

The original M2pepBiotin is degraded in mouse serum within 4 h via N-terminal degradation and endolytic cleavages at the W/W site in the binding region and at the S/K site in the spacer region. By replacing L-lysines in the spacer region with D-lysines and cyclizing the binding region of the peptide, we demonstrate significant improvement in serum stability of M2pep. Nonetheless, the cyclized peptide was eventually degraded by prolonged incubation in mouse serum for more than 1 day. Since cyclization of the first generation cyclized M2pep(RY)Biotin was based on a disulfide bridge which may be prone to linearization over time due to changes in the redox environment, we investigated whether the alternative cyclization chemistries can further improve serum stability over disulfide cyclization. Serum stability of the newly-synthesized cyclic M2pep(RY)Biotin peptides were evaluated by incubation in mouse serum and analysis for the presence of peptides at different time points by MALDI-ToF MS. As observed for Disulfide-cyclized M2pep(RY)Biotin27, Amide-, Triazole-, and DFBP-cyclized M2pep(RY)Biotin were stable in serum for at least 24 h after which degradation products were observed (FIG. 17A-C). Multiple attempts to quantify the extent of degradation were unsuccessful due to variability in the amount of extracted peptides following the protein precipitation with ACN, thus preventing quantitative comparisons regarding serum stability. Nonetheless, by identifying the degradation products, it is clear that degradation was initiated at either Y/W or W/Y which corresponds to the W/W site of the original M2pepBiotin. Even though endolytic cleavage at the Y/W/Y site seems to be the rate-determining step towards peptide degradation of cyclic M2pep(RY)Biotin, the new generation analogs are expected to be more favorable than the disulfide-cyclized analog due to insensitivity to reduction. Surprisingly for DFS-cyclized M2pep(RY)Biotin, formation of the “−20 Da” side product was observed over time (FIG. 17D). This side product was also observed when cyclization reaction with DFS was left for too long but was not observed in the purified peptide stock solution in water, PBS, or PBSA for at least one day (Figure S5). Similarly, the Pentelute lab has recently reported an observation on oxidative degradation when DFS is used to cyclize cysteine-containing peptides. The group has also demonstrated superior stability when DFS is instead used to cyclize lysine-containing peptides. Hence, studies by others as well as ours emphasize the need to evaluate any newly developed cyclization chemistries for stability towards physicochemical and physiological stresses such as redox environment and enzymatic degradation. Since we have shown here a higher serum stability of DFBP-cyclized M2pep(RY)Biotin as well as a similar improvement in binding affinity of DFBP- and DFS-cyclized M2pep(RY), it is possible to utilize the DFS-stapled peptide phage library reported by the Derda lab for initial screening of high affinity peptide candidates before synthesizing the peptide with DFBP cyclization for the follow-up evaluation. All in all, different cyclization chemistries as well as robust and flexible synthetic strategies greatly expand our toolbox for peptide optimization that is needed for the development of new effective targeting agents or therapeutics.

### REFERENCES

1. Siegel R L, Miller K D, Jemal A. Cancer Statistics, 2015. CA Cancer J Clin. 2015; 65:5-29.
2. Allen T M. Ligand-targeted therapeutics in anticancer therapy. Nat Rev Cancer. 2002; 2:750-63.
3. Sanna V, Pala N, Sechi M. Targeted therapy using nanotechnology: Focus on cancer. Int J Nanomedicine. 2014; 9:467-83.
4. Bartlett D W, Su H, Hildebrandt I J, Weber W a, Davis M E. Impact of tumor-specific targeting on the biodistribution and efficacy of siRNA nanoparticles measured by multimodality in vivo imaging. Proc Natl Acad Sci USA. 2007; 104:15549-54.
5. Chari R V J, Miller M L, Widdison W C. Antibody-drug conjugates: An emerging concept in cancer therapy. Angew Chemie—Int Ed. 2014; 53:3796-827.
6. Srinivasarao M, Galliford C V., Low P S. Principles in the design of ligand-targeted cancer therapeutics and imaging agents. Nat Rev Drug Discov. 2015; 14:203-19.
7. Khoury G a., Smadbeck J, Kieslich C a., Floudas C a. Protein folding and de novo protein design for biotechnological applications. Trends Biotechnol. 2014; 32:99-109.
8. Brown K C. Peptidic tumor targeting agents: the road from phage display peptide selections to clinical applications. Curr Pharm Des. 2010; 16:1040-54.
9. Yu M K, Park J, Jon S. Targeting strategies for multifunctional nanoparticles in cancer imaging and therapy. Theranostics. 2012; 2:3-44.
10. Bertrand N, Wu J, Xu X, Kamaly N, Farokhzad O C. Cancer nanotechnology: The impact of passive and active targeting in the era of modern cancer biology. Adv Drug Deliv Rev. 2014; 66:2-25.
11. Kim J-W, Kim T-D, Hong B S, Kim O Y, Yoon W-H, Chae C-B, et al. A serum-stable branched dimeric anti-VEGF peptide blocks tumor growth via anti-angiogenic activity. Exp Mol Med. 2010; 42:514-23.
12. Akcan M, Stroud M R, Hansen S J, Clark R J, Daly N L, Craik D J, et al. Chemical re-engineering of chlorotoxin improves bioconjugation properties for tumor imaging and targeted therapy. J Med Chem. 2011; 54:782-7.
13. Ngambenjawong C, Cieslewicz M, Schellinger J G, Pun S H. Synthesis and evaluation of multivalent M2pep peptides for targeting alternatively activated M2 macrophages. J Control Release. 2016; 224:103-11.
14. Cieslewicz M, Tang J, Yu J L, Cao H, Zavaljevski M, Motoyama K, et al. Targeted delivery of proapoptotic peptides to tumor-associated macrophages improves survival. Proc Natl Acad Sci USA. 2013; 110:15919-24.
15. Gajewski T F, Schreiber H, Fu Y-X. Innate and adaptive immune cells in the tumor microenvironment. Nat Immunol. 2013; 14:1014-22.
16. Whiteside T L. The tumor microenvironment and its role in promoting tumor growth. Oncogene. 2008; 27:5904-12.
17. Lamagna C, Aurrand-Lions M, Imhof B a. Dual role of macrophages in tumor growth and angiogenesis. J Leukoc Biol. 2006; 80:705-13.
18. Zheng J-S, Tang S, Qi Y-K, Wang Z-P, Liu L. Chemical synthesis of proteins using peptide hydrazides as thioester surrogates. Nat Protoc. 2013; 8:2483-95.
19. Cochran R, Cochran F. Phage display and molecular imaging: expanding fields of vision in living subjects. Biotechnol Genet Eng Rev. 2010; 27:57-94.
20. McGregor D P. Discovering and improving novel peptide therapeutics. Curr Opin Pharmacol. 2008; 8:616-9.
21. Pollaro L, Heinis C. Strategies to prolong the plasma residence time of peptide drugs. Medchemcomm. 2010; 1:319-24.
22. Walensky L D, Bird G H. Hydrocarbon-Stapled Peptides: Principles, Practice, and Progress. J Med Chem. 2014; 57:6275-88.
23. Sahu a, Soulika a M, Morikis D, Spruce L, Moore W T, Lambris J D. Binding kinetics, structure-activity relationship, and biotransformation of the complement inhibitor compstatin. J Immunol. 2000; 165:2491-9.
24. Nguyen L T, Chau J K, Perry N a., de Boer L, Zaat S a J, Vogel H J. Serum stabilities of short tryptophan- and arginine-rich antimicrobial peptide analogs. PLoS One. 2010; 5:1-8.
25. Liu G W, Livesay B R, Kacherovsky N a., Cieslewicz M, Lutz E, Waalkes A, et al. Efficient Identification of Murine M2 Macrophage Peptide Targeting Ligands by Phage Display and Next-Generation Sequencing. Bioconjug Chem. 2015; 26:1811-7.
26. Bordo D, Argos P. Suggestions for “safe” residue substitutions in site-directed mutagenesis. J Mol Biol. 1991; 217:721-9.
27. Gallivan J P, Dougherty D a. Cation-pi interactions in structural biology. Proc Natl Acad Sci USA. 1999; 96:9459-64.
28. Dougherty D a. Cation-{pi} Interactions Involving Aromatic Amino Acids. J Nutr. 2007; 137:15045-1508.
29. Hughes R M, Wiggins K R, Khorasanizadeh S, Waters M L. Recognition of trimethyllysine by a chromodomain is not driven by the hydrophobic effect. Proc Natl Acad Sci USA. 2007; 104:11184-8.
30. Clark R J, Fischer H, Dempster L, Daly N L, Rosengren K J, Nevin S T, et al. Engineering stable peptide toxins by means of backbone cyclization: stabilization of the alpha-conotoxin MII. Proc Natl Acad Sci USA. 2005; 102:13767-72.
31. Kolodziej A F, Zhang Z, Overoye-Chan K, Jacques V, Caravan P. Peptide optimization and conjugation strategies in the development of molecularly targeted magnetic resonance imaging contrast agents. Methods Mol Biol. 2014; 1088:185-211.
32. Laoui D, Movahedi K, Van Overmeire E, Van den Bossche J, Schouppe E, Mommer C, et al. Tumor-associated macrophages in breast cancer: distinct subsets, distinct functions. Int J Dev Biol. 2011; 55:861-7.

We claim:

1. A peptide comprising contiguous amino acid residues according to the formula:
X0-X1-D-P*-W-X2-X3-X4-X5-W*-X6-X7
wherein
X0 is absent or an N-terminal functional group;
X1 is 1-7 contiguous amino acid residues;
P* is P or hydroxyproline;
X2 is a single amino acid;
X3 is a single amino acid;
X4 is a single amino acid;
X5 is a single amino acid;
W* is W or w;
X6 is 1-6 contiguous amino acid residues; and
X7 is absent, KKK, or kkk,
wherein when X1 is YEQ, X0 is an N-terminal functional group;
wherein the peptide is linear or is a cyclic peptide; and
wherein when the peptide is a cyclic peptide, one residue of X1 is covalently bound to one residue of X6, optionally through a linker L.

2. A peptide comprising contiguous amino acid residues according to the formula:
X91-X92-X93-X94-X95-X96-X97-X98-X99
wherein
X91 is 0-2 contiguous amino acid residues;
X92 is a single amino acid;
X93 is a single amino acid;
X94 is 0-1 contiguous amino acid residues;
X95 is a single amino acid;
X96 is a single amino acid;
X97 is 0-4 contiguous amino acid residues;
X98 is a single amino acid; and
X99 is 0-5 contiguous amino acid residues; and
and wherein at least one of the following applies:
X92 is W, X93 is P, and X96 is D;
X93 is P, X95 is S, and X98 is A;
X93 is P, X95 is S, and X98 is L; or
X92 is W, X93 is V, X96 is D, and X98 is W.

3. The peptide of claim 2, comprising contiguous amino acid residues according to the formula:
X11-W-P-X12-D-X13-X14-X15
wherein
X11 is 0-2 contiguous amino acid residues;
X12 is a single amino acid;
X13 is 0-3 contiguous amino acid residues;
X14 is a single amino acid; and
X15 is 0-4 contiguous amino acid residues.

4. The peptide of claim 2, comprising contiguous amino acid residues according to the formula:
X21-X22-P-X23-S-X24-X25-X26-X27-X28
wherein
X21 is 0-1 contiguous amino acid residues;
X22 is a single amino acid;
X23 is 0-1 contiguous amino acid residues;
X24 is a single amino acid;
X25 is a single amino acid;
X26 is 0-3 contiguous amino acid residues;
X27 is a single amino acid; and
X28 is 0-5 contiguous amino acid residues.

5. The peptide of claim 2 comprising contiguous amino acid residues according to the formula:
X41-W-V-X42-D-X43-W-X44
wherein
X41 is 0-2 contiguous amino acid residues;
X42 is a single amino acid;
X43 is 0-3 contiguous amino acid residues; and
X44 is 0-3 contiguous amino acid residues.

6. The peptide of claim 1, wherein the peptide comprises an amino acid sequence selected from the group consisting of:
(SEQ ID NO: 05)
Ac-YEQDPWGVKWWYGGGSKKK;
(SEQ ID NO: 06)
X0-YEQDPWGVKwWYGGGSKKK;
(SEQ ID NO: 07)
X0-YEQDPWGVKwwYGGGSKKK;
(SEQ ID NO: 08)
X0-YEQDPWGVKPWYGGGSkkk;
(SEQ ID NO: 09)
X0-YEQDPWGVKYWYGGGSkkk;
(SEQ ID NO: 10)
X0-YEQDPWGVRWWYGGGSKKK;
(SEQ ID NO: 11)
X0-YEQDPWGVRYWYGGGSkkk;
(SEQ ID NO: 12)
X0-YEQDPWGVKZaWYGGGSkkk;
(SEQ ID NO: 13)
X0-YEQDPWGVRYWyGGGSkkk;
(SEQ ID NO: 14)
X0-YEQDZbWGVRYWyGGGSkkk;
and
(SEQ ID NO: 15)
X0-RYEQDPWGVRYWyGGGSkkk;
wherein Ac is an acetyl moiety, Za is P, D, T, R, or H, and Zb is hydroxyproline.

7. The peptide of claim 1, wherein the peptide comprises an amino acid sequence selected from the group consisting of: 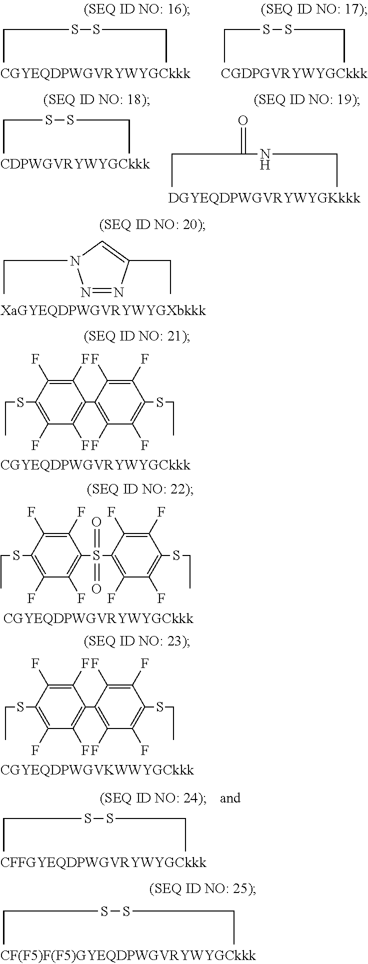
wherein Xa is azidolysine, Xb is propargylglycine; and F(F5) is pentafluorophenylalanine.

8. The peptide of claim 3, wherein the peptide comprises an amino acid sequence selected from the group consisting of:
(SEQ ID NO: 26)
WPTDHQMLRIPM;
(SEQ ID NO: 27)
WPWDPLRISDWL;
(SEQ ID NO: 28)
LPWPSDQIILMW;

9. The peptide of claim 4, wherein the peptide comprises an amino acid sequence selected from the group consisting of:
(SEQ ID NO: 29)
TYPSTQWFFAKF;
(SEQ ID NO: 30)
YPSSEQLLAWWG;
(SEQ ID NO: 31)
FFPSEQVLIAAL;
(SEQ ID NO: 32)
ELPSVEQLWDFF;
(SEQ ID NO: 33)
NAPSIYDWLATL;
(SEQ ID NO: 34)
KLPSPYDLYLFL;
(SEQ ID NO: 35)
GLPSSAELERLW;
(SEQ ID NO: 36)
LPSSAELLWALR;
(SEQ ID NO: 37)
RLPTSMELLAAF;
(SEQ ID NO: 38)
TWVSDLDMWLGA;
and
(SEQ ID NO: 39)
SYWVPDIVWAGL.

10. The peptide of claim 5, wherein the peptide comprises TWVSDLDMWLGA (SEQ ID NO:38) or SYWVPDIVWAGL (SEQ ID NO:39).

11. A peptide comprising the peptide of claim 1 coupled to a therapeutic agent.

12. An M2 macrophage-binding agent comprising the peptide of claim 1.

13. A nucleic acid encoding the peptide of claim 1.

14. A pharmaceutical composition, comprising a polypeptide of claim 1, and a pharmaceutically acceptable carrier.

15. A polymer comprising two or more repeating units that form a backbone, wherein the repeating units comprise at least one unit comprising a conjugated M2 macrophage-binding peptide and optionally one or more unconjugated units.

16. The polymer of claim 15, wherein at least one of the conjugated M2 macrophage-binding peptides comprises contiguous amino acid residues according to the formula:
X0-X1-D-P*-W-X2-X3-X4-X5-W*-X6-X7
wherein
X0 is absent or an N-terminal functional group;
X1 is 1-7 contiguous amino acid residues;
P* is P or hydroxyproline;
X2 is a single amino acid;
X3 is a single amino acid;
X4 is a single amino acid;
X5 is a single amino acid;
W* is W or w;
X6 is 1-6 contiguous amino acid residues; and
X7 is absent, KKK, or kkk,
wherein when X1 is YEQ, X0 is an N-terminal functional group;
wherein the peptide is linear or is a cyclic peptide; and
wherein when the peptide is a cyclic peptide, one residue of X1 is covalently bound to one residue of X6, optionally through a linker L.

17. The polymer of claim 15, wherein at least one of the conjugated M2 macrophage-binding peptides comprises contiguous amino acid residues according to the formula:
X11-W-P-X12-D-X13-X14-X15
wherein
X11 is 0-2 contiguous amino acid residues;
X12 is a single amino acid;
X13 is 0-3 contiguous amino acid residues;
X14 is a single amino acid; and
X15 is 0-4 contiguous amino acid residues.

18. The polymer of claim 15, wherein at least one of the conjugated M2 macrophage-binding peptides comprises contiguous amino acid residues according to the formula:
X21-X22-P-X23-S-X24-X25-X26-X27-X28
wherein
X21 is 0-1 contiguous amino acid residues;
X22 is a single amino acid;
X23 is 0-1 contiguous amino acid residues;
X24 is a single amino acid;
X25 is a single amino acid;
X26 is 0-3 contiguous amino acid residues;
X27 is a single amino acid; and
X28 is 0-5 contiguous amino acid residues.

19. The polymer of claim 15, wherein at least one of the conjugated M2 macrophage-binding peptides comprises contiguous amino acid residues according to the formula:
X41-W-V-X42-D-X43-W-X44
wherein
X41 is 0-2 contiguous amino acid residues;
X42 is a single amino acid;
X43 is 0-3 contiguous amino acid residues; and
X44 is 0-3 contiguous amino acid residues.

20. A method of treating or ameliorating a subject suffering from cancer comprising: administering to the subject a therapeutically effective dose of a peptide according to claim 1, or a polymer comprising two or more repeating units that form a backbone, wherein the repeating units comprise at least one unit comprising a peptide according to claim 1, wherein the peptide or polymer are coupled to a therapeutic agent, thereby delivering the therapeutic agent and treating or ameliorating the cancer.
